(19) United States
(12) Patent Application Publication
(10) Pub. No.: US 2025/0061733 A1
(43) Pub. Date: Feb. 20, 2025

(54) Method for providing source data for training or validating processing model for processing analyte image sequences

(71) Applicant: Carl Zeiss Microscopy GmbH, Jena (DE)

(72) Inventors: Manuel Amthor, Jena (DE); Daniel Haase, Zöllnitz (DE); Ralf Wolleschensky, Jena (DE)

(21) Appl. No.: 18/807,391

(22) Filed: Aug. 16, 2024

(30) Foreign Application Priority Data
Aug. 18, 2023 (DE) 10 2023 122 213.9

(51) Int. Cl. G06V 20/69; G06V 10/141; G06V 10/46; G06V 10/56; G06V 10/762; G06V 10/774; G06V 10/776; G06V 20/70; G16B 30/00; G16B 40/20

(52) U.S. Cl. CPC G06V 20/698; G06V 10/141; G06V 10/46; G06V 10/762; G06V 10/774; G06V 10/776; G06V 20/695; G06V 20/70; G16B 30/00; G16B 40/20; G06V 10/56

(57) ABSTRACT

A method for providing source data for training and validating a processing model for processing analyte image sequences. An analyte image sequence is generated by labeling analytes with markers in a plurality of coloring rounds and detecting the markers with a camera. The markers are selected, in particular based on a codebook, such that signal sequences of analytes in an image region over the analyte image sequence comprise colored signals and uncolored signals, in particular an order of the colored and uncolored signals is based on the codebook. The camera captures an image of the analyte image sequence in each of the coloring rounds. The method comprises capturing a spot analyte image sequence of a sample, and comprises providing an evaluation of image regions based on image signals of the image regions. Further, the method comprises identifying spot image regions from the evaluation of the image regions.

### RELATED APPLICATIONS

This application claims priority to German Patent Application No. 10 2023 122 213.9, filed on Aug. 18, 2023, which is incorporated herein by reference in its entirety.

### BACKGROUND OF THE INVENTION

EP 2 992 115 B1 discloses a method for identifying analytes by coloring the analytes to be identified with markers in a plurality of coloring rounds. The markers consist of oligonucleotides and dyes coupled thereto, which are generally fluorescent dyes. The oligonucleotides are specific for specific portions of the analytes to be identified. However, the individual oligonucleotides of the markers are not unique for the respective analytes. However, on account of the plurality of coloring rounds, it is possible to carry out an unambiguous determination of the analytes since a plurality of different markers can be assigned to a specific oligonucleotide after the execution of the plurality of coloring rounds and the assigned plurality of markers are then unique for the respective analytes.

With this method, a wide variety of analytes can be detected in vitro, for example in a cell, by means of a fluorescence microscope. The analytes can be an RNA, in particular an mRNA or a tRNA. The analytes can also be a portion of a DNA.

A multiplicity of analytes which can be identified in parallel with the coloring rounds explained above are often located in a sample, even if these are different analytes. The more analytes are located in the sample, the greater the number of markers to be detected in the respective coloring rounds. In the case of an automatic capturing and evaluation of the corresponding image signals, the image signals of all markers in the sample must be captured and also distinguished from image signals in the sample which are not caused by markers coupled to analytes.

WO 2020/254519 A1 and WO 2021/255244 A1 disclose a further method with which, inter alia, analytes, for example also proteins, can be identified. In this method, probes which are specific for the respective analytes are firstly coupled thereto. The probes have oligonucleotide residues which do not hybridize with the analytes. Decoding oligonucleotides which have a supernatant relative to the free residues are hybridized to the free oligonucleotide residues. Marker molecules are hybridized to a dye at the supernatants. Also in this method, a sequence of image signals is generated at the respective analytes in a plurality of coloring rounds, which provide information about the respective analyte present. However, methods are also known in which the marker molecules bind directly to the free oligonucleotide residues.

DE 10 2022 131 444.8 describes a plurality of methods for processing analyte image sequences, wherein analytes are coupled to marker molecules on images of the analyte image sequences in a plurality of coloring rounds and the analytes can be identified from an order of the marker molecules coupled to the analytes. Different processing models are used in particular for processing the analyte image sequences.

### SUMMARY OF THE INVENTION

In practice, it has been shown that a reading out of source data or the providing of an annotated data set, with which a processing model for processing analyte image sequences can be trained, from recorded analyte image sequences is very computationally complex, since a suitable matching with a codebook, which comprises target signal sequences for each of the occurring analyte types of a respective experiment, has to be carried out in each case for all image points of a recorded analyte image sequence in order to detect or identify signal sequences of analytes in order to read out a corresponding signal sequence after the identification as part of the ground truth.

The invention is based on the object of providing a method for providing source data for training a processing model for processing analyte image sequences, wherein the method requires considerably less computation capacity or computation power in order to provide the source data in comparison with the known methods for providing source data.

A further object of the invention is to provide an annotated dataset based on the source data, wherein the annotated dataset is designed for training processing models for processing analyte image sequences.

A further object of the invention is to provide a method for processing analyte image sequences, wherein the processing is carried out using a processing model which has been trained for processing the analyte image sequence by means of the annotated dataset, the annotated data set being based on the source data.

A further object of the invention is to provide a method for validating processing models, wherein the method for validating uses the annotated dataset.

A further object of the invention is to provide an evaluation device for evaluating images of an analyte image sequence for carrying out one of the abovementioned methods.

One aspect of the invention relates to a method for providing source data for training and validating a processing model for processing analyte image sequences. An analyte image sequence is generated by labeling analytes with markers in a plurality of coloring rounds and detecting the markers with a camera. The markers are selected, in particular based on a codebook, such that signal sequences of analytes in an image region over the analyte image sequence comprise colored signals and uncolored signals, in particular an order of the colored and uncolored signals is based on the codebook. The camera captures an image of the analyte image sequence in each of the coloring rounds. The method comprises capturing a spot analyte image sequence of a sample, and comprises providing an evaluation of image regions based on image signals of the image regions. Further, the method comprises identifying spot image regions in the spot images from the evaluation of the image regions, wherein the evaluation of spot image regions of spot images for colored signals has a spot evaluation. Further, the method comprises capturing one or more source analyte image sequences comprising source images registered to the spot images, comprising reducing a scene contrast such that the image signals of the spot image regions in the source images of at least one of the source analyte image sequences for colored signals has a non-spot evaluation, and providing at least the source analyte image sequence as source data.

The source data preferably also comprise the spot analyte image sequence.

According to the present invention, source data are data of a physical system which are necessary for preparing an annotated dataset, in particular these can be, for example, image data recorded in an experiment with a recording device, for example an image capturing device, but also any other form of measuring device. In particular, the source data per se can already represent an annotated data set with input data and target data, but the source data can also comprise data for which the target data still has to be determined.

According to the present invention, an analyte is a substance whose presence or absence in a sample is intended to be specifically detected and which is intended to be encoded in the case of its presence. This can be any type of entity, including a protein, polypeptide, protein or a nucleic acid molecule (e.g. RNA, PNA or DNA), also referred to as transcript. The analyte offers at least one location for specific binding with analyte-specific probes. An analyte in the sense of the invention can comprise a complex of objects or articles, for example at least two individual nucleic acid, protein or peptide molecules. In one embodiment of the disclosure, an analyte excludes a chromosome. In another embodiment of the disclosure, an analyte excludes DNA. In some embodiments, an analyte can be an encoding sequence, a structural nucleotide sequence or a structural nucleic acid molecule, in particular a nucleotide sequence which is translated into a polypeptide, generally via mRNA, if it is under the control of suitable regulatory sequences. The boundaries of the encoding sequence are determined by a translation start codon at the 5′ terminus and a translation stop codon at the 3′ terminus. An encoding sequence can comprise genomic DNA, cDNA, EST and recombinant nucleotide sequences, but is not limited thereto. Depending on which type of analyte is to be identified, such methods are referred to, for example, as spatial transcriptomy or also as multiomy.

According to the present invention, the regions of the electromagnetic spectrum, for short spectral regions, which each comprise a color of a marker, are also referred to as color channels. The images separated into the color channels are monochromatic images and contain an image signal of the image point as a value or measured value for each image point.

In the following, a marker is understood to mean a molecule which can be coupled, in particular hybridized, to analytes of the respective analyte type with an oligonucleotide specific for the respective analyte type, referred to as probe oligonucleotide. Furthermore, the markers have an oligonucleotide with a dye coupled thereto, referred to as decoding oligonucleotide. The probe oligonucleotide and the decoding oligonucleotide can be coupled directly to one another or to one another via further connecting oligonucleotides. According to the present invention, a marker always comprises at least the probe oligonucleotide and the decoding oligonucleotide. A coupling and decoupling of a marker with an analyte can be carried out either completely or in a plurality of steps. In particular, only parts of the marker can also always be coupled or decoupled between different coloring rounds or different marking rounds.

The term image signal is understood below to mean either the value or measured value of an image point of the image for a specific color or a specific color channel, or the image signal comprises values of different primary colors of a color space of a multicolor image.

The term image sequence is understood below to mean that an image sequence comprises a plurality of images of a sample, wherein the images at least partially map the same section of a sample.

The term analyte image sequence is understood below to mean that an analyte image sequence is an image sequence, wherein analytes occurring in the sample are labeled with markers during the recording of the analyte image sequence according to a codebook, wherein different analyte types are labeled such that the analyte type can be identified on the basis of the codebook on the basis of a signal sequence of an analyte over the analyte image sequence.

The term signal sequence is understood below to mean that the signal sequence comprises the image signals of image regions of an image sequence, wherein the image regions of the different images of the image sequence are preferably registered to one another or are registerable to one another. In images registered to one another, the image regions of the images registered to one another capture the signals of the same location in the sample. The signal sequence of an image region comprises the image signals of the images of the image sequence of the respective image region. According to the present invention, a signal sequence of an image region over an image sequence is accordingly a sequence of signals captured from the same location in the sample in the images of the image sequence.

A signal sequence relates in each case to a specific image sequence, if a plurality of image sequences are recorded, there are corresponding signal sequences for each image sequence. In particular, a signal sequence can be based on an analyte image sequence.

According to the present invention, the image regions each comprise for example only one image point, an area of contiguous image points or a contiguous volume in an image stack.

According to the present invention, the image signals of neighboring image points of an image region can be combined, for example an image signal of the image region is determined based on the image signals of the image points of the image region, which image signal represents the image signals of the image points of the image region. The image signal of the image region can be for example a maximum value, a minimum value, a mean value, a median value or another representative value for the image signals of the image points of the image region.

As a result of the fact that a plurality of image points are combined to form an image region, a required computation power can be reduced during the evaluation of image signals of analyte image sequences.

According to an alternative, the image signals of an image region can also comprise all image signals of all image points of the image region.

By contrast, a pixel-by-pixel evaluation possibly enables a separation of signals of analytes lying close together which, when combining the plurality of image points to form an image region, would merge with one another with only a single value and could no longer be separated from one another.

Correspondingly, the size of an image region can be selected depending on an expected analyte density in the sample. Preferably, the size of an image region can vary over the entire image, depending in each case on the analyte density expected in the image region.

As a result of the fact that the size of an image region is selected depending on an expected analyte density, a required computation power can be optimized corresponding to an expected analyte density.

According to the present invention, the processing by means of a processing model comprises inputting an input datum into the processing model and outputting an output datum by the processing model. Accordingly, the processing of analyte image sequences by means of a processing model comprises inputting an input datum based on the analyte image sequences into the processing model.

According to the present invention, a processing model is a model configured for processing input data and for outputting output data. The processing model can be a classic model which has been created, for example, by means of classic optimization or analysis methods, equally well the processing model can be a model trained by means of a learning method.

According to the present invention, a machine learning model, in particular a neural network, is a processing model which can be trained, in particular, by means of a supervised or unsupervised learning process for processing input data and outputting output data.

In particular, a processing model according to the present invention can be configured for processing analyte image sequences, then it is also referred to as an analyte image sequence processing model. However, the present invention also relates to processing models which are not restricted to processing only analyte image sequences.

The processing according to the present invention can comprise a wide variety of processing mappings.

Whether the processing models described are trained or classic models or models specifically configured for processing analyte image sequences can, in each case, be derived from the context or is explained accordingly.

According to the present invention, the training of a processing model is understood to mean supervised learning or unsupervised learning, in particular self-supervised learning.

During supervised learning, an annotated dataset is used. The annotated data set comprises input data and target data, wherein an annotation or identification, referred to as target datum, of the target data corresponds to each input datum of the input data.

The target datum is a datum used in the training of the processing model for executing a processing mapping, to which an output datum output by the processing model on the basis of the input datum is to be adapted. The approximation is carried out with the aid of an objective function. The objective function is in particular a gain or loss function which predefines how differences between the output datum of the processing model and the target datum are evaluated. The evaluation of the differences can be carried out on the basis of the entries of the data by entry or by a comparison of more abstract structures.

The loss function can capture, for example, differences between the output datum and the predefined target datum. If the input datum and the target datum are, for example, images, the comparison can be carried out pixel by pixel. The pixel by pixel differences can be added in absolute value (as absolute values) in an L1 loss function. The square sum of the pixel by pixel differences is formed in an L2 loss function. To minimize the loss function, the values of model parameters of the processing model are changed, which can be calculated, for example, by gradient descent and back propagation.

During unsupervised learning, as is used, for example, in autoencoders for training, the training data comprise only the input data, but no target data.

An evaluation can be provided, in particular, automatically according to specific evaluations, but, for example, also semi-automatically, in which a user carries out an evaluation of the spot images. This is easily possible, in particular, since the spot image regions in the spot images can be easily identified with the naked eye. The evaluation can be carried out, for example, in binary fashion, in which either the evaluation of spot image region or non-spot image region is assigned to each image region. However, the evaluation can also be carried out by outputting continuous evaluation values, wherein a spot evaluation is then an evaluation above a spot threshold value.

According to the present invention, the spot threshold value is selected precisely such that image signals which lie above the spot threshold value are with high probability image signals of a colored signal of an agglomerating analyte. The spot threshold value is determined in particular for each image region or for neighboring image regions together; in the case of specific samples, the spot threshold value can also be determined over an entire spot image, but this is rather the exception. The spot threshold value lies, for example, five standard deviations above the average image signal of the background image regions surrounding an image region to be evaluated. In particular, the spot threshold value lies, for example, ten or fifteen standard deviations above the average image signal of the surrounding background image regions. The spot threshold value is very similar in each case in particular in subareas of the sample with a similar autofluorescence background.

In typical analyte image sequences recorded by the multiomy, some of the analytes to be detected agglomerate in the sample, i.e. in the examined sample there are accumulation points at which a concentration of analytes of an analyte type is so high that the image signals of colored signals of the image regions capturing agglomerating analytes, also referred to as spot image regions, agglomeration image regions, agglomeration analyte image regions, have a particularly high image signal, i.e. are particularly bright. In the images of the image sequence, these spot image regions appear so bright that they can be easily identified with the naked eye. In particular with respect to the respective image background.

The signal sequences of the spot image regions can be read out from the recorded analyte image sequences, for example, by means of a simple threshold value comparison, and one of the analyte types can be assigned to each of the read-out signal sequences by matching with a codebook. The signal sequence and the assigned analyte type can then be used as ground truth for the training of a processing model for finding and identifying image signals of agglomerating analytes of the respective analyte type in an analyte image sequence.

Alternatively, the spot image regions can, however, also be identified, for example, with a classic blob detection (blob detection).

The agglomerating analytes make up only a small percentage of the analytes typically contained in a sample. The far predominant proportion of analytes typically contained in samples are non-agglomerating analytes. Since the image signals of image regions capturing the colored signals of non-agglomerating analytes, also called non-spot analyte image regions, differ only very weakly from image signals of background image regions surrounding the non-spot analyte image regions, the identification of the non-spot analyte image regions is unequally more difficult since, instead of a simple threshold value valid over, for example, an entire microscope image, the image signals of non-spot analyte image regions corresponding to the colored signals each have to be identified locally, for example, from the image signals of the respectively surrounding image regions. If it is furthermore also taken into account that the data volume which arises during the recording of the analyte image sequence is very high with up to several terabytes per experiment, it follows immediately therefrom that a correspondingly high time and computation requirement is required in order to analyze the data of all image points as described above.

The inventors have recognized that, on the basis of the spot analyte image sequences, it is possible to generate source analyte image sequences which comprise signal sequences which are so similar to the signal sequences of non-spot analyte image regions over an analyte image sequence that it is possible to use them to train processing models to process the signal sequences of non-spot analyte image regions in an analyte image sequence, i.e. in particular to identify and analyze the signal sequences of non-spot analyte image regions.

For this purpose, first of all, after the recording of a spot image of the spot analyte image sequence in the recorded spot image, all image regions which possibly capture image signals of the agglomerating analytes, the so-called spot image regions, must be identified. If the spot image regions are identified, one or more source images with a scene contrast reduced compared to the spot images can be captured by suitable illumination of the sample, wherein for at least one of the source images the image signals of the spot image regions in the source images differ only slightly from the image signals of the background image regions surrounding the spot image regions. The source images then have a greatly reduced dynamic range, also referred to as scene contrast, compared to the spot images, such that the image signals of the spot image regions in the source images for colored signals lie in particular below the spot threshold value or the evaluation is precisely a non-spot evaluation.

If the scene contrast of the sample and thus the dynamic range of the source images is sufficiently reduced compared to the spot images, the resulting source signal sequences of the spot image regions can no longer be distinguished from signal sequences of non-spot analyte image regions in the spot images. On the basis of the spot image regions identified in the spot images, source signal sequences of the spot image regions can be read out from the source analyte image sequence over the source analyte image sequence, the source signal sequences then comprise the image signals of the spot image regions over the coloring rounds and can be used in the training of a processing model for processing analyte signal sequences, wherein the processing model trained by means of the source signal sequences thus read out is accordingly also trained for processing signal sequences of non-spot analyte image regions or is trained to also process them. The source analyte image sequence thus comprises source signal sequences from which an annotated dataset for training a processing model for processing analyte image sequences can be provided, wherein the trained processing model can also process, i.e. in particular identify and analyze, signal sequences of non-agglomerating analytes.

Whereas in the prior art, for all image regions of an analyte image sequence which possibly capture signals of analytes whose image signals have to be analyzed in order also to locate signal sequences of non-spot analyte image regions in the image sequence and to provide the respective signal sequences with corresponding annotation as annotated data set, the method according to the present invention makes it possible, by virtue of the fact that the source data comprising the source signal sequences are generated on the basis of the easily identifiable spot image regions by reducing the scene contrast, to considerably reduce the time and computational effort for providing the source data for generating an annotated dataset. The source data can also be recorded selectively during the recording of a sample in a so-called sacrificial subarea of the sample, so that source data can also be provided directly for each new experiment, from which source data annotated datasets can then be generated in a simple manner for the training of a wide variety of processing models.

The recording of analyte image sequences of the sample preferably comprises exciting a fluorescence of the markers by means of a light source and detecting a fluorescence signal emitted by the excited markers.

As a result of the fact that fluorescent markers are used during the capturing of the analyte image sequences, the reducing of the scene contrast can be affected in a particularly simple manner, for example by suitable spectral filters.

Preferably, n of the coloring rounds respectively correspond to a marking round, wherein the markers coupled to the different analytes in a marking round have decoding oligonucleotides with n different dyes, wherein analytes of a specific analyte type are labeled with a maximum of a specific one of the n dyes in a marking round and are therefore detected in a maximum of one of the n coloring rounds of a marking round. The n different dyes are respectively recorded in a different color channel, also referred to as color contrast or fluorescence contrast.

The n different dyes in each of the marking rounds are preferably the same n dyes. According to an alternative, different dyes can also be used in different marking rounds. A different number of dyes can also be used in each marking round.

Preferably, a total of n×m=k coloring rounds are carried out and n×m=k images are recorded. A signal sequence thus comprises k image signals, wherein each analyte type has a colored signal in a maximum of m of the coloring rounds.

The image signals of spot image regions for colored signals for at least one of the source images are preferably above a background threshold value and below an autofluorescence spot threshold value.

When recording analyte image sequences, autofluorescence spot image regions also occur repeatedly, i.e. image regions which capture signals of autofluorescent subareas of the sample which are brighter than a normal autofluorescent background, also called autofluorescence spots here. These image regions have an image signal significantly below the spot threshold value in the respective region of the sample, but also significantly above an average image signal of an autofluorescent background of the sample. By lowering the scene contrast for at least one of the source images to such an extent that the image signals of the spot image regions of the at least one source image lie below the autofluorescence spot threshold value, a processing model can also be trained for identifying the source signal sequences if the image signals of the source signal sequences of colored signals lie only between the average image signals of the background and the image signals of the autofluorescence spots. Autofluorescence spots can be identified in that they always have approximately the same signal level in the case of the same fluorescence contrast and corresponding to the same configuration of the imaging device; a specific order of colored and uncolored signals corresponding to a codebook as in the case of analytes does not occur in the case of such autofluorescence spots.

In particular, the image signals of the autofluorescence spots recover more quickly during the bleaching than the image signals of the spot image regions.

The image signals of such autofluorescence spots are typically below the spot threshold value.

By virtue of the fact that the scene contrast of the source images is reduced to such an extent that the image signals of spot image regions are indeed still above a background threshold value but below an autofluorescence spot threshold value, the signal sequences based on the image signals of the spot image regions can be used as source data for training processing models for processing analyte image sequences, wherein a processing model trained with these data is trained by means of these data to also process signal sequences of non-agglomerating analytes.

The method preferably also comprises recording a plurality of source analyte image sequences, wherein the image signals of the source image regions of the plurality of source analyte image sequences each lie below the image signals of the spot image regions.

In particular, a spot configuration of an imaging device is preferably used during the recording of the source analyte image sequence and a source configuration of the imaging device is preferably used during the recording of the source analyte image sequence.

In addition to the agglomerating analytes which generate image signals with evaluations above the spot threshold value in image regions, in the case of multiomy in many samples there are always also analytes which generate image signals in image regions which although lying below the spot threshold value, the image signals of which nevertheless lie significantly above an image signal of a background. So that intermediate signal sequences can also be generated for these intermediate image regions, further source analyte image sequences can also be recorded, which then serve as training data in order to be able to find these intermediate image regions with processing models as well.

The reducing of the scene contrast preferably comprises bleaching at least one or more spot subareas or spot subareas of the sample corresponding to the spot image regions.

The bleaching preferably comprises illuminating the spot subareas of the sample multiple times, recording a plurality of source analyte image sequences and/or applying a chemical fluorescence suppressor.

As a result of the fact that the spot image regions are bleached by suitably repeated illumination or by means of a chemical fluorescence suppressor, the source analyte image sequence can be recorded in a particularly simple manner.

A number of illuminations in the illuminating multiple times is preferably a predefined number of illuminations, the number of recorded source analyte image sequences or the number of source images is a predefined number of recorded source analyte image sequences or the number of illuminations and the number of recorded source analyte image sequences are determined from the image signals of the spot image regions and are determined in particular based on the image signals of the spot image regions, the spot threshold value and/or an average background image signal.

Depending on the examined sample, a bleaching behavior of the sample can be quite different. If the bleaching behavior of the examined sample is known, for example, on the basis of repeated examinations of comparable samples, a number of illuminations in the illuminating multiple times can be predefined, but if, for example, a new sample is examined, the bleaching behavior of which is not yet known, an evaluation or a comparison of the image signals of the spot image regions can be carried out after each recording of an image or after each illumination which has been carried out and it is possible to decide on the basis of the evaluation or on the basis of the comparison, for example with the spot threshold value, how often the sample still has to be or will be illuminated in order to obtain suitable image signals for a source signal sequence. If, therefore, a known sample is examined, for example, the illuminating multiple times can be carried out without recording further images and only one source image is recorded after or with the last illumination. If, on the other hand, a hitherto unexamined sample with unknown bleaching behavior is examined, then a source image can be recorded in each case after each illumination or, for example, after each second illumination, it can then be determined on the basis of the image signals in the respective spot image regions whether the sample is sufficiently bleached out or further illuminations have to be carried out.

The present invention thus provides a method according to which it is possible to react flexibly to the bleaching behavior of examined samples in order to record an optimal source-analyte image sequence according to the bleaching behavior of the sample.

The bleaching preferably comprises matching image signals of an autofluorescence of background image regions surrounding the spot image regions and the image signals of the spot image regions. The matching is carried out such that the image signals of the background image regions, generally autofluorescence signals of the sample, are reduced less strongly, i.e. bleached, than the image signals of the spot image regions.

By carrying out the bleaching such that the image signals of the background image regions are attenuated less strongly, it can be achieved that in the source images the image signals of the autofluorescence approximately correspond to the image signals of the autofluorescence in the spot images, while the image signals of the analytes, in particular the image signals corresponding to the colored signals, are greatly reduced by the bleaching. As a result, it is achieved that the signal sequences of the image regions which capture the image signals of the bleached agglomerating analytes have similar signal levels to the signal sequences of the non-spot analyte image regions.

Preferably, the matching in particular comprises one or more of:
1 selecting a temporal distance between two successive illuminations such that the image signals of the autofluorescence regenerate between two successive illuminations, illuminating the sample during the bleaching such that only the spot subareas of the sample are illuminated, wherein in particular the light source is a laser and an imaging device is a laser scanning microscope, LSM,
processing the image signals of the background image regions such that the image signals of the background image regions remain approximately constant over all illuminations,
selecting a concentration of the chemical fluorescence suppressor such that the image signals of colored signals of spot image regions approach the image signals of the background image regions.

Because a temporal distance between two successive illuminations is suitably selected, the image signals of the autofluorescence of the background image regions can regenerate more strongly between the two illuminations than the image signals of the spot image regions regenerate. As a result of the suitable selection of the temporal distance, it is thus achieved that the scene contrast of the sample is reduced, as a result of which source signal sequences of spot signal sequences of image regions capturing agglomerating analytes can be generated which are identical to the signal sequences of the non-spot analyte image regions.

As a result of the fact that only the spot image regions are selected and these are selectively illuminated with a laser, source signal sequences of spot signal sequences of image regions capturing agglomerating analytes can likewise be generated which resemble signal sequences of non-spot analyte image regions.

As a result of the fact that a suitable concentration of a chemical fluorescence suppressor is used during the bleaching, source signal sequences of spot signal sequences which resemble signal sequences of non-spot analyte image regions can be generated in a simple manner.

The reducing of the scene contrast preferably comprises changing the spectral properties of an imaging device, in particular the recording of the source analyte image sequence comprises using a source configuration of the imaging device, wherein the source configuration has changed spectral properties compared to a spot configuration of the imaging device used during the recording of the spot analyte image sequence, such that a scene contrast of the source analyte image sequence is reduced compared to the spot analyte image sequence.

The source configuration and the spot configuration are preferably different for different ones of the coloring rounds, in particular the spot configuration and the source configuration are dependent on a marker to be detected in the respective coloring round.

As a result of the fact that the scene contrast of the sample is reduced by means of simple adaptation of the spectral properties of the imaging device, for example by changing spectral filters, signal sequences which correspond to signal sequences of non-spot analyte image regions can in turn be generated by identifying spot image regions. The method thus constitutes a simple possibility of generating source data for an annotated dataset for training a processing model for processing analyte image sequences.

Preferably, the spectral filtering comprises at least one or a combination of:
adapting an illumination spectrum emitted by the light source,
adapting chromatic properties of a light source filter for filtering the illumination spectrum emitted by the light source,
adapting chromatic properties of a fluorescence filter for filtering the fluorescence signal of the markers, and/or
adapting chromatic properties of a dichroic mirror for adapting a spectral component which is reflected and/or transmitted by the dichroic mirror.

As a result of the fact that suitable parts of a spectrum used during the recording of the analyte image sequences are filtered during the spectral filtering such that the scene contrast of the sample is reduced, the method according to the present invention can generate signal sequences from spot signal sequences in a simple manner which resemble the signal sequences of non-spot analyte image regions in order thus to provide source data for an annotated dataset for training a processing model for processing analyte image sequences.

Preferably, the source configuration and the spot configuration differ in one or more of the following spectral properties:
an illumination spectrum of the light source,
a spectral transmittance of a light source filter for filtering the illumination spectrum emitted by the light source,
a spectral transmittance of a fluorescence filter for filtering the fluorescence signal of the markers, and/or
a spectral transmittance of a dichroic mirror, wherein the spectral transmittance indicates a spectral component which is reflected and/or transmitted by the dichroic mirror.

Preferably, a source illumination spectrum used according to the source configuration has a lower overlap with the excitation spectrum of the marker to be excited than a spot illumination spectrum of the light source used according to the spot configuration, such that a scene contrast of the source analyte image sequence is reduced compared to the spot analyte image sequence, wherein in particular the image signals of the spot image regions of the source images are reduced compared to the image signals of the spot image regions of the spot images.

As a result of the fact that the source illumination spectrum covers only a smaller proportion of the excitation spectrum of the marker to be excited, the fluorescence of the marker is excited to a lesser extent, for which reason a generated fluorescence intensity is reduced compared to an excitation with the spot illumination spectrum. Thus, signal sequences which resemble the signal sequences of non-spot analyte image regions can be generated in a simple manner from spot signal sequences by means of the method according to the present invention in order thus to provide source data for an annotated dataset for training a processing model for processing analyte image sequences.

Preferably, a source light source filter for filtering the illumination spectrum of the light source used according to the source configuration filters the illumination spectrum of the light source such that the resulting filtered illumination spectrum has a lower overlap with the excitation spectrum of the marker to be excited than an illumination spectrum filtered with a spot light source filter used according to the spot configuration, such that a scene contrast of the source analyte image sequence is reduced compared to the spot analyte image sequence, wherein in particular the image signals of the spot image regions of the source images are reduced compared to the image signals of the spot image regions of the spot images.

As a result of the fact that a part of the illumination spectrum of the light source is cut off by means of the source light source filter during the recording of the source analyte image sequence, the scene contrast of the sample can be reduced, whereby signal sequences which resemble the signal sequences of non-spot analyte image regions can be generated in a simple manner from the spot signal sequences in order thus to provide source data for an annotated dataset for training a processing model for processing analyte image sequences.

Preferably, a source fluorescence filter used according to the source configuration has a worse overlap with the fluorescence spectrum of the excited marker than a spot fluorescence filter used according to the spot configuration, such that a scene contrast of the source analyte image sequence is reduced compared to the spot analyte image sequence, wherein in particular the image signals of the spot image regions of the source images are reduced compared to the image signals of the spot image regions of the spot images.

As a result of the fact that a part of the fluorescence spectrum of the marker to be detected is cut off by means of the source fluorescence filter during the recording of the source analyte image sequence, the scene contrast of the sample can be reduced, whereby signal sequences which resemble the signal sequences of non-spot analyte image regions can be generated in a simple manner from the spot signal sequences in order thus to provide source data for an annotated dataset for training a processing model for processing analyte image sequences.

Preferably, chromatic properties of a dichroic source mirror used according to the source configuration and a dichroic spot mirror used according to the spot configuration are selected such that the source mirror has a worse match with the illumination spectrum, the excitation spectrum of the marker to be excited and/or the fluorescence spectrum of the excited marker than the spot mirror, such that a scene contrast of the source analyte image sequence is reduced compared to the spot analyte image sequence, wherein in particular the image signals of the spot image regions of the source images are reduced compared to the image signals of the spot image regions of the spot images.

As a result of the fact that the chromatic properties of the dichroic mirror are suitably varied during the recording of the source analyte image sequence, the scene contrast of the sample can be reduced, whereby signal sequences which resemble the signal sequences of non-spot analyte image regions can be generated in a simple manner from the spot signal sequences in order thus to provide source data for an annotated dataset for training a processing model for processing analyte image sequences.

The reducing of the scene contrast preferably comprises unequally illuminating the sample for capturing the source images, wherein agglomeration sample areas of the sample mapping to the spot image regions, also referred to as spot subareas, and non-agglomeration sample areas of the sample not mapping to spot image regions, also referred to as background subareas, are just illuminated unequally such that the scene contrast is reduced.

As a result of the fact that agglomeration sample areas and non-agglomeration sample areas of the sample are illuminated differently, the scene contrast of the sample during the capturing of source images and a light loading of the sample during the capturing of the source images can be reduced in a simple manner.

The unequal illumination preferably comprises a stronger illumination of the non-agglomeration sample areas, a weaker illumination of the agglomeration sample areas or both.

The light source is preferably in particular a laser during the unequal illumination and the unequal illumination comprises controlling the laser such that the sample is illuminated evenly during the recording of the spot images and the agglomeration sample areas and the non-agglomeration sample areas of the sample are illuminated differently during the recording of the source images, as a result of which either image signals in the image regions detecting non-agglomeration sample areas are increased, the image signals in the image regions detecting agglomeration sample areas are reduced or both.

The reducing of the scene contrast of the sample before the recording of the source analyte image sequence preferably comprises one or more of the bleaching described above, the changing of the spectral properties and the unequal illumination.

By virtue of the fact that the spectral filtering, the bleaching and the unequal illumination can be combined in order to reduce the scene contrast, the loading of the sample when recording the source data or when providing the annotated data set can be reduced.

Preferably, the providing of an evaluation of the spot image regions comprises one or more of:
matching the image signals of image regions of a spot image with a spot threshold value and evaluating image regions as having the spot evaluation if image regions have image signals above the spot threshold value,
inputting the image signals of image regions of at least one spot image into a candidate identification model, wherein the candidate identification model outputs an evaluation or an evaluation value for each image region.

As a result of the fact that the spot image regions can be identified by comparison with the spot threshold value, the identification is particularly simple.

As a result of the fact that the spot image regions are identified by means of the candidate identification model, the candidate spot image regions can be identified particularly quickly and efficiently.

The method preferably further comprises determining the spot threshold value, in particular based on an average image signal in the spot image, based on the image region to be evaluated and an average image signal in a surrounding of the image region, based on context information or based on a coloring round.

As a result of the fact that the spot threshold value is respectively determined only locally in the surrounding of the respective image region, spots can be reliably identified, in particular in the case of different intensities of the autofluorescence or of the background signal.

The candidate identification model is preferably a machine learning model which has been trained to evaluate the image signals or the image regions, wherein image signals of image regions of a spot image with an evaluation above the spot threshold value are identified as the spot image regions, wherein the candidate identification model takes into account in particular the image signals of the image region to be evaluated and the image signals of image regions surrounding the image region to be evaluated in the evaluation.

Due to the fact that the candidate identification model is implemented as an evaluation model, a user receives, with evaluations determined by the evaluation model, a value which is easy to verify or interpret and on the basis of which a proper functioning of the evaluation model can be verified.

The candidate identification model has preferably also been trained to determine the spot threshold value from the image signals of the spot image.

As a result of the fact that the candidate identification model also determines the spot threshold value, the evaluation model can also be trained for changing illumination conditions in a wide variety of experiments in which an absolute brightness may vary greatly, but the image signals of the spot image regions are offset sufficiently from the image signals of the background image regions. A single candidate identification model can thus be trained for many different applications.

The candidate identification model is preferably designed as a detection model, wherein the detection model has been trained to output a list of the spot image regions with the spot evaluation.

According to the present invention, a detection model is a machine learning model which has been trained to identify predetermined detection patterns in input data and to output a list, on the basis of which the identified detection patterns can be read out or determined and further processed. In particular, the list is a list of localizations, for example a localization in the input data. The input data can be in particular an image, an image stack or else an input tensor. The exact format of the localization depends in particular on the input data and the detection patterns to be identified, in particular on the format of the input data and the detection pattern to be identified.

By virtue of the fact that the candidate identification model is designed as a detection model, the outputs of the detection model can be further processed directly. This considerably simplifies the further processing of the data.

The candidate identification model is preferably designed as a classification model.

According to the present invention, classification models have been or are trained to assign a class to an input datum. The output datum can be in particular a class assigned to the input datum, wherein the format can be in particular a vector, wherein each entry of the vector corresponds precisely to one of the possible classes to be assigned and in particular a “1” entry in the vector indicates the class. Alternatively, a class number can also be output, for example. Alternatively, however, a classification model can also be trained such that it outputs a vector, wherein the entries in the vector each indicate a probability that the respective input datum belongs to the class corresponding to the entry of the vector. Depending on an implementation, the respective format of the output datum of classification models and accordingly also the format of the target datum in an annotated data set for training the classification model varies.

According to the present invention, the input datum can be an individual signal sequence based on the analyte image sequence, for example the image signals of an individual signal sequence over the analyte image sequence of an individual image region can be input into the processing model as input datum. If signal sequences of individual image regions are input into the model individually and successively, it is said that the receptive field of the model then comprises only a single image region; alternatively, however, the receptive field of the model can also comprise signal sequences of neighboring image regions. The model then processes the signal sequence of the respective image region inter alia on the basis of the image signals or signal sequences of the further image regions in the receptive field. It is also said that the spatial context is included in the processing of the image signals or the signal sequence of the image region, in this case the image signals or signal sequences of the neighboring image regions which belong to the receptive field of the model.

A number of the image regions in the receptive field can be selected, for example, on the basis of the point spread function of the microscope, with the result that a diameter of the receptive field is not greater than, only insignificantly greater than or, for example, twice as large as a diameter of a region onto which a point in a sample is mapped on the basis of the point spread function. For example, the receptive field is 3×3, 5×5, 7×7, 9×9, 13×13, 17×17 image regions in size; however, the receptive field can also be 3×3×3, 5×5×5, 7×7×7, 9×9×9, 13×13×13 or else 17×17×17 image regions in size if image stacks are recorded in the coloring rounds.

If the candidate identification model is implemented as a classification model, the output datum comprises in particular an identified class, i.e. whether it is an image region or not.

According to a further alternative, the candidate identification model can also be trained to carry out semantic segmentation.

Semantic segmentation in the sense of the present invention is an image-to-image mapping which assigns an output value corresponding to a semantic to each image point or each image region of an input datum, i.e. the output datum is also an image again, the output image is also referred to as semantic segmentation mask or only segmentation mask.

For example, a semantic learned by a candidate identification model can be that a class of “spot image region” or “non-spot image region” is assigned to each image region, wherein each of the classes corresponds to a specific value. In the case of correspondingly more complex semantics, the output datum has more/more different output values in the output datum in accordance with a number of classes in the semantics.

The method preferably further comprises determining the spot configuration and/or the source configuration for recording the spot images and/or the source images by means of a configuration determination model.

As a result of the fact that a configuration determination model is used for determining the source configuration and the spot configuration, the source configuration and/or the spot configuration can be determined in a simple manner according to the available components or the markers used.

The input data of the configuration determination model preferably comprise context information of the spot analyte image sequence to be recorded, in particular information about an excitation spectrum of markers used, an emission spectrum of the markers used, as well as available optical components, in particular light sources, light source filters, fluorescence filters and/or dichroic mirrors.

Preferably, the determining of the source configuration comprises inputting the spot images into the configuration determination model, wherein the configuration determination model has been trained to determine a source configuration for recording the source image based on an input spot image, wherein the source configuration comprises one or more of the following information:
a source light source which is used during the recording of the source image,
a source light source filter which is used during the recording of the source image,
a source fluorescence filter which is used during the recording of the source image,
an illuminance of the source light source,
a number of illuminations carried out,
a number of recorded source images,
a temporal distance between successive illuminations,
a size and/or position of an illuminated light section, and/or
a concentration of a chemical fluorescence suppressor.

As a result of the fact that a configuration determination model determines the source configuration for recording the source image, the source configuration for recording the source images can be determined in a simple manner in each case by input of the respective spot image. Thus, the source configuration for recording the source images can be determined individually for each sample, as a result of which the recording of the source images can be carried out particularly efficiently and gently for the sample.

Preferably, context information is also input into the configuration determination model for determining the source configuration.

Preferably, the determining of a source configuration further comprises inputting a source image into the configuration determination model and determining, by the configuration determination model, the source configuration for further source images to be recorded based on the input source image or based on the input source image and the spot image.

As a result of the fact that not only spot images but also source images can be used when determining the source configuration, a determination of the source configuration can be further improved, whereby the sample can in turn be preserved, because additional recordings can be avoided, for example. Such a case occurs, for example, if the successively recorded source images are in each case input into the configuration determination model in a new sample with an unknown bleaching behavior, then the configuration determination model learns the same behavior a little better with each input, whereby a loading of the sample can be reduced.

Preferably, context information is included in the evaluation of the images.

By virtue of the fact that the context information is taken into account during the evaluation, a configuration of the recording device can be taken into account during the evaluation, for example, whereby the evaluations become more exact.

The method preferably also comprises reading out candidate analyte signal sequences from spot image regions of the spot analyte image sequence, wherein signal sequences of image regions for each coloring round comprise image signals of the image regions of the image of the respective coloring round, and determining a result class of the candidate analyte signal sequences, wherein the result classes comprise at least one class for each analyte type to be identified, and the determination of the result class is carried out on the basis of a codebook.

The result classes preferably also comprise a background class for candidate analyte signal sequences, which cannot be assigned to any of the analyte types to be identified, and in particular candidate analyte signal sequences assigned to the background class are identified as non-analyte signal sequences, and the candidate analyte signal sequences assigned to one of the analyte types are identified as analyte signal sequences.

According to the present invention, a codebook indicates for each analyte type which of the markers couple to the analytes of the respective analyte type in which of the respective coloring rounds. In particular, the codebook can indicate for each of the analyte types and each of the coloring rounds whether and, if so, which markers are to be coupled to the analytes of the analyte type in the respective coloring rounds.

By virtue of the fact that a respective analyte type is determined for the candidate spot signal sequences found prior to the selecting of source data for the annotated data set, or it is determined whether the candidate analyte signal sequences are actually analyte signal sequences, that is to say signal sequences which are based on image signals of analytes, the respectively determined analyte type can be taken into account in the selecting of the source data for the annotated data set, such that the corresponding source signal sequences can be selected on the basis of the analyte type of the analyte signal sequences, wherein source signal sequences are preferably respectively selected for all analyte types to be identified. The present invention thus provides a method for generating an annotated dataset in which source signal sequences for training can be provided for all analyte types to be identified.

Preferably, the method further comprises determining registration information of the images of the different coloring rounds with respect to one another, wherein the registration information comprises at least one of translation information and rotation information of the images of the different coloring rounds with respect to one another.

The registration information comprises at least translation and/or rotation information, wherein the translation and rotation information each indicate a translation or rotation of an image to another in order to align the specific point clouds of the images to be registered to one another.

In addition to the translation and rotation information, the registration information can also be information about, for example, a linear global transformation, representable by a transformation matrix which also comprises affine transformations and shears, and information about local transformations, global or local non-linear transformations and intrinsic camera parameters (e.g. distortion correction, numerical or chromatic aperture of the camera).

According to an alternative, the registration information can respectively be output for each image, or registered images are calculated and only the registered images are stored. If the registration information is stored in addition to the images, this has the advantage that the registration information can still be verified subsequently, even if the storing of the registration information takes additional memory.

Preferably, the registration information is determined based on the spot images of the different coloring rounds or the registration information is determined based on the spot images and a part of the source images of the different coloring rounds.

The registration information for the spot images and the source images are preferably identical.

As a result of the fact that the images of different coloring rounds are registered to one another and the source images are respectively recorded in the respective coloring round, it is not necessary to additionally determine registration information for the source images, whereby a computational effort can be reduced when registering the source images with respect to one another. As a result of the higher scene contrast of the spot images, a quality during the determining of the registration information can furthermore be improved.

The determining of registration information preferably comprises:
identifying registration structures in the images to be registered, wherein in particular images with a sufficient scene contrast are used,
determining location information of the identified registration structures, and
determining the registration information based on the location information and the identified registration structures.

As a result of the fact that firstly the registration structures in images with a sufficient scene contrast are identified, the determining of the registration information can be carried out significantly more exactly and due to the higher scene contrast of the spot images than if, for example, the source images with a minimum scene contrast were used here.

According to the present invention, a sufficient scene contrast is a scene contrast in which spot image regions or the spot image regions are offset by a minimum amount from the background image regions.

For example, the image signal of the image regions used differs by, for example, one, two, three, four or else five standard deviations from the image signals of background image regions, wherein the standard deviation of the mean value of the image signals of background image regions is used.

Preferably, the registration structures in particular comprise one or more of the following structures:
cell edge or cell edges,
cell organelles,
cell nuclei,
cytoskeleton,
mitochondria,
image regions with a brightness above a brightness threshold, in particular spot image regions with an evaluation above the spot threshold value,
point objects, caused by markers or marker agglomerations,
cover glasses,
structures with a minimum degree of image sharpness, for example edges,
image regions which have a certain, in particular predetermined, structure or texture,
holding frame,
sample stages,
sample holder,
corrugated plates/corrugated dishes, and/or
non-sample structures.

Whereas, in the prior art, bright spots are used above all for registering images of an image sequence to one another, the present method can register to one another a multiplicity of structures appearing in recorded images.

The determination of the registration information is preferably carried out on the basis of the location information, in particular by means of an iterative nearest point algorithm.

The determining of the registration information preferably comprises inputting the images of the different coloring rounds into a registration model, wherein the registration model has been trained to identify registration structures in the images, to determine the registration information or to identify the registration structures and to determine the registration information based on the registration structures, wherein the registration model has in particular been trained to identify the registration structures in the images.

According to an alternative, the registration model can also directly determine the registration information. In this case, the registration model is directly trained to register the microscope images of an image sequence to one another. Such a model is also called end-to-end registration model.

An annotated data set with which the end-to-end registration model is trained comprises, for example, a set of microscope images not registered to one another as input data and a set of microscope images registered to one another as target data. Alternatively, instead of the microscope images registered to one another, the annotated data set can comprise only the registration information as target data.

As a result of the fact that a registration model is trained to identify the registration structures, the registration structures can be found particularly quickly and efficiently.

The registration model preferably identifies the registration structures based on semantic segmentation of the spot images and/or of the source images or of a part of the source images carried out by the registration model, and the registration is carried out on the basis of the semantic segmentation of the semantically segmented images.

The registration model is preferably also trained to determine the registration information.

As a result of the fact that a registration model is used for determining registration structures and for determining the registration information, it is possible, during the registration of the spot images of the spot analyte image sequence to one another, not only to use particularly bright image regions for the registration of the images to one another, as is customary in the prior art, but the registration of the images to one another can also be carried out on the basis of the abovementioned extended registration structures. This improves the continuity of the registration over the entire area of the images in comparison with a registration of images by means of point clouds known from the prior art.

The registration model is preferably in particular trained to output the location information of the registration structures.

As a result of the registration model outputting the location information of the registration structures, a user can check the registration model simply and intuitively.

The registration model is preferably in particular trained such that the location information of the registration structures is output as an intermediate output and the registration information is output based on the outputs if an output layer of the registration model can be determined, wherein the location information is in particular semantic segmentations of the microscope images input and the semantic segmentations of the different microscope images are then aligned by the registration model by determining the registration information.

As a result of the registration model outputting the location information of the registration structures as an intermediate output, this intermediate output can be used in training as a deep supervision signal, on the basis of which the training of the registration model can be improved.

Preferably, the inputting of the images of the different coloring rounds comprises, for each coloring round, a spot image and a plurality of source images, wherein the plurality of source images are source images successively recorded in a coloring round with image signals decreasing in the candidate source image regions.

As a result of the fact that source images with sufficiently high image signals in specific image regions are used in the source images which are used for the registration, source images can also be used during the registration, since the registration structures can still be well identified here as well. As a result of the number of images which are used during the determining of the registration information being increased, the statistics and thus also a resolution or an accuracy during the determining of the registration information are improved, for example because the registration information can be determined with an accuracy of less than one image point.

A further aspect of the present invention relates to a method for providing an annotated dataset for training a processing model, the method comprising:
selecting input data for inputting into a processing model from source data, wherein the source data has been recorded according to the method described above,
selecting target data, wherein the target outputs are determined depending on the processing model to be trained, and
providing the input data and the assigned target outputs as annotated data set.

According to the present invention, the input data is based on the source analyte image sequence and the target data is determined on the basis of the source analyte image sequence and the spot analyte image sequence.

The target datum is the output datum desired upon input of the input datum into the processing model. The processing model is trained by means of the annotated dataset to carry out a virtual processing mapping to be learned by a machine learning system comprising the processing model.

For providing the annotated dataset, it is possible to use different ones of the captured image data of the source analyte image sequence and, if appropriate, of the spot analyte image sequence. Depending on a processing model used, an annotated dataset can thus be created from the source data. The invention accordingly makes it possible to provide an annotated dataset for a wide variety of processing models based on a single set of source data, in particular the source data with the source analyte image sequences comprise signal sequences which resemble the signal sequences of non-spot analyte image regions in order thus to provide source data for an annotated dataset for training a processing model for processing analyte image sequences.

The processing model is preferably a registration model. The registration model is trained to determine registration information of at least two images from different coloring rounds. The selecting of input data comprises selecting at least two images of different coloring rounds of the spot analyte image sequence at least partially capturing the same parts of the sample and the selecting of target data in particular comprises reading out registration information from the source data or the selecting of target data comprises determining registration information for the at least two spot images and selecting the registration information as target datum.

In this case, the determination of the registration information is carried out analogously to the determination of the registration information described above with reference to the method for providing the source data.

As described above, a processing model can also be used for determining registration information; correspondingly, an annotated dataset can be created from the source data, with which a registration model of this type can be trained.

The at least two images preferably comprise either spot images or source images or both, wherein the image signals of image regions of selected source images lie above a background threshold value.

According to the present invention, the background threshold value is sufficiently above an average value of image signals of background image regions. Sufficiently above means here, for example, one, two, three, four or five standard deviations above the average value.

As a result of the fact that source images with sufficiently high image signals in specific image regions are used in the source images which are used for the registration, source images can also be used during the registration, since the registration structures can still be well identified here as well. As a result of the number of images which are used during the determining of the registration information being increased, the statistics and thus also a resolution or an accuracy during the determining of the registration information are improved, for example because the registration information can be determined with an accuracy of less than one image point.

The processing model is preferably a candidate extraction model which has been trained to extract candidate signal sequences from an analyte image sequence. The input data of such a candidate extraction model comprises signal sequences of spot image regions over the source analyte image sequence.

The candidate extraction model can preferably be implemented as a classification model, semantic segmentation model, detection model or else as an image-to-image model.

According to the implementation, these different models have different input data or output data, and in training, the target data must be selected according to the output data.

According to the present invention, a candidate extraction model is configured to extract signal sequences of image regions from an image sequence, the image regions of which capture signals of analytes with a high probability. For the training of such candidate extraction models, a sufficient number of signal sequences are required both of image regions which actually capture analytes and of background image regions. The annotated dataset for the training of such candidate extraction models can be generated in a simple manner from the recorded source data.

The processing model is preferably a result class assignment model which has been trained to assign signal sequences to a result class. Such a result class assignment model uses signal sequences of image regions of the source analyte image sequence as input data. The result classes to be assigned comprise at least one class for each analyte type to be identified. Furthermore, there should also be one or at least one class for image regions of background image regions, a so-called background class.

The result class assignment model can likewise be implemented in different implementations, for example as a classification model, as an embedding model, as a semantic segmentation model, as a detection model or as a binarization model.

The classification model assigns an input datum to a result class, here to the analyte types or the background class, and outputs a corresponding output datum. As described above, the class can be output directly; this is also called hard assignment, or a probability distribution over the result classes to be identified is output; this is also called soft assignment.

The semantic segmentation model is designed such that a result class is assigned to each image region, that is to say, for example, the respective analyte type or the background class.

The detection model is trained to output a list of image regions which capture analytes, and to output the specific result class, that is to say the specific analyte type, for the output image regions.

The binarization model is trained to output a binarized signal sequence, wherein the binarization model assigns a binary value to the image signals of a signal sequence, that is to say the binarization model decides whether the respective image signal is the image signal of a colored analyte or the image signal of a non-colored analyte, for short a colored signal or an uncolored signal. On the basis of the resulting bit sequence, that is to say the binarized signal sequence, matching with target signal sequences of a codebook can now be carried out, the analyte type whose target signal sequences have the most matches with the binarized signal sequence is assigned to the signal sequence. If, on the other hand, a number of matching bit values is less than a threshold value, then no analyte type is assigned, instead it is decided that the respective signal sequence or the respective image region is to be assigned to the background, that is to say the background class.

The embedding model embeds an input signal sequence into an embedding space such that the target embedding with a minimum distance to the embedding of the input signal sequence can be determined on the basis of a distance to the target embeddings of target signal sequences, and the respective analyte type is assigned according to the minimum distance.

As a result of the fact that source data and signal sequences with reduced scene contrast are generated according to the method described above, a processing model can be trained to assign signal sequences of analytes even if optical identification of the image regions on the basis of the image signals is not possible. The generation of an annotated dataset with conventional means as described above is associated with a high time and computational effort since all signal sequences of all image points or all image regions have to be correspondingly binarized in order to match them with target signal sequences. This is already associated with a considerable memory and computation effort solely on account of the high data volumes which occur during the recording of such analyte image sequences. As a result of the fact that the source analyte image sequences are generated by corresponding reduction of the scene contrast, the processing model can also be trained to find and classify signal sequences of non-spot analyte image regions for which the image signals of colored signals do not lie above the spot threshold value.

The selection of the input data is preferably carried out on the basis of the determined target data such that a sufficient number of input data is present in the annotated data set for all result classes to be identified.

In order to have sufficient training examples for each of the result classes to be identified, a sufficient number of input data must be selected for each of the result classes to be identified for the annotated data set. This enables training which is as uniform as possible and of high quality, respectively, and thus ensures good identification for all result classes to be identified.

Source signal sequences can preferably be generated by interchanging the order of the image signals of the source signal sequences.

For example, if a sufficient number of source signal sequences could not be recorded for specific analyte types with particular specific orders of the colored and uncolored signals, further source signal sequences can be generated in order thus to enable uniform training for all analyte types.

The selection of input data for the annotated data set is preferably carried out on the basis of the determined result classes of the signal sequences of the spot analyte image sequence and the input data is selected from the source analyte image sequence.

As a result of the input data being selected from the source analyte image sequences, the respective processing model is trained to also identify image regions which capture non-agglomerating analytes.

Preferably, the input data comprise, depending on the processing model to be trained, one or more of:
of the source analyte image sequences,
the spot analyte image sequence,
images or fields of view of the source analyte image sequences,
one or more source signal sequences from the source analyte image sequences, wherein the source signal sequences are signal sequences of spot image regions of the source images over the coloring rounds,
one or more background signal sequences, wherein the background signal sequences comprise signal sequences of background image regions, signal sequences of image signals randomly selected from the source analyte image sequence or signal sequences of image signals randomly selected from an unlabeled analyte image, and wherein the background image regions are image regions that capture no image signals of analytes, and wherein the unlabeled analyte images are images in which analytes are not labeled with markers, and wherein image regions of the unlabeled analyte images capture autofluorescence signals of the sample.

As a result of the suitable selection of the input data for the annotated data set, the respective processing model can be reliably trained both for identifying analyte signal sequences and for identifying background signal sequences.

Preferably, the determining of the target data comprises inputting context information into the processing model.

Preferably, context information is stored during the recording of the spot analyte image sequence as well as during the recording of the source analyte image sequence.

Preferably, context information is included in the identifying of spot image regions.

Preferably, context information is included in the reducing of a scene contrast.

Preferably, the context information comprises one or more of:
information about an excitation spectrum of a marker used,
information about available and/or used light sources and/or their exposure spectra,
information about available and/or used light source filters and/or their chromatic properties in the spectral filtering of the illumination spectrum of the light source,
information about available and/or used fluorescence filters for spectrally filtering the fluorescence spectrum emitted by the sample,
information about available and/or used dichroic mirrors and their chromatic properties,
an illuminance of the light source,
an illumination time of the light source,
a number of illuminations,
a number of recorded second images,
a temporal distance between two successive illuminations,
a size and/or position of an illuminated field of view,
a concentration of a fluorescence suppressor in the sample,
a type of the recorded sample,
a type of sample carrier which was used for recording the sample image, for example whether a chamber slide, a microtitre plate, a slide with cover glass or a Petri dish was used,
image recording parameters, such as, for example, information about illuminance, illumination time, filter settings, fluorescence excitation, contrast method, or sample table settings,
information about objects contained in the respective microscope image,
application information which indicates for which type of application the microscope images were recorded,
information about a user who has recorded the images of the sample.

The use of context information can be advantageous in a multiplicity of the methods described above. If, for example, information about the excitation spectrum of used or available light sources and their illumination spectrum, about chromatic properties of available or used filters, for example light source filters or fluorescence filters, as well as about chromatic properties of used or available dichroic mirrors is known in an imaging device, the different information can be adapted to one another such that, for example, a sample is illuminated only minimally, whereby a light loading in the sample can be minimized.

The same applies to the illuminances of the light sources and available illumination times of the light sources.

If, for example, the number of illuminations or the number of recorded source images is in each case known, then, for example, the same behavior of certain samples can be determined and predicted better in subsequent experiments, whereby a loading of the examined sample can in turn be reduced.

If context information with regard to a type of the recorded sample is respectively recorded together with the recorded image sequences, then, for example, samples with similar properties can be identified on the basis of this context information in later experiments and, for example, annotated datasets can be combined for these samples or pre-trained models with similar properties can be provided.

If, for example, information about objects contained in the respectively recorded image is stored as context information, then, for example, in the analysis in doubt, it can be determined on the basis of the context information whether specific analytes can appear at specific locations or not.

A further aspect relates to a method for training a machine learning system having a processing model for processing analyte image sequences, wherein analyte image sequences are generated by labeling analytes with markers in a plurality of coloring rounds and detecting the markers with a camera, the camera captures an image of the analyte image sequence in each coloring round, the markers are selected such that image signals of an analyte in an image region over the analyte image sequence comprise colored signals and uncolored signals, the method comprising:
providing an annotated data set,
optimizing an objective function by adapting the model parameters of the processing model, wherein the objective function captures a difference between an output datum output by the processing model on the basis of an input datum of the annotated data set and a target datum of the annotated data set corresponding to the input datum, characterized in that the annotated data set was generated with the aid of the method described above.

As a result of the fact that an annotated dataset which was generated with the method described above is used in the method for training a machine learning system having a processing model, the processing model can be trained to also find or identify or classify analytes in image sequences which cannot be identified with the naked eye or with conventional methods, for example methods which use threshold values in order to identify bright spots of agglomerating analytes. The present invention thus provides a method for training a machine learning system with a processing model with improved sensitivity.

A further aspect relates to a method for training a machine learning system having a processing model for processing analyte image sequences, wherein analyte image sequences are generated by labeling analytes with markers in a plurality of coloring rounds and detecting the markers with a camera, the camera captures an image of the analyte image sequence in each coloring round, the markers are selected such that image signals of an analyte in an image region over the analyte image sequence comprise colored signals and uncolored signals, the method comprising:
providing a training data set,
carrying out a cluster analysis algorithm with the processing model on the basis of the training data set, characterized in that the training data set is based on the source data generated by means of the method described above, in particular on the signal sequences of the spot image regions of the source analyte image sequence.

The source data obtained can also be used for unsupervised training, in particular for a cluster analysis algorithm. In particular by using the signal sequences of the spot image regions of the source analyte image sequences, the cluster analysis can be trained such that signal sequences of non-spot analyte image regions can also be reliably processed.

A further aspect of the invention relates to an annotated dataset for use in a method for training a machine learning system comprising a processing model, wherein the annotated dataset has been provided by means of the method described above and is used in particular in the method described above for training a machine learning system having a processing model.

By virtue of the fact that the annotated dataset as generated in the method described above is used, a processing model or a machine learning system having a processing model with improved sensitivity can be provided as described above.

A further aspect of the present invention relates to a method for validating a processing model for processing an image sequence, wherein the image sequence is generated by labeling analytes with markers in a plurality of coloring rounds and detecting the markers with a camera, the camera captures an image of the image sequence in each coloring round, the markers are selected such that signal sequences of image regions that capture image signals of an analyte comprise colored signals and uncolored signals over the image sequence. The camera captures an image of the image sequence in each coloring round, the markers are selected such that signal sequences of image regions that capture image signals of an analyte comprise colored signals and uncolored signals over the image sequence. The method comprises providing an annotated dataset for validating the processing model, inputting the annotated dataset into the processing model and matching result outputs output by the processing model with target outputs contained in the annotated dataset, characterized in that the annotated dataset was generated by means of the method described above.

According to this aspect of the invention, the annotated dataset generated with the method described above serves as a standard, on the basis of which other evaluation methods for processing image sequences can be validated. Since signal sequences with image signals of non-agglomerating analytes are also contained in the annotated dataset, it is possible to validate by means of the method how sensitively a processing model can identify such non-agglomerating analytes.

Preferably, the matching of the output data with the target data comprises one or more of:
matching of correctly identified analyte image regions,
matching of location information assigned to analyte image regions,
number of identified analyte image regions or analyte image regions identified per analyte type,
number of image regions incorrectly identified as analyte image regions,
number of analyte image regions incorrectly assigned to the background.

The validating preferably comprises comparing a plurality of different processing models or processing models trained with different annotated data sets with one another.

The validation preferably comprises validating with different ones of the source analyte image sequences, wherein a sensitivity of the respectively validated processing model is carried out on the basis of the decreasing image signals in the successively recorded source analyte image sequences.

A further aspect of the present invention relates to a computer program product comprising instructions which, when the program is executed by a computer, cause the latter to carry out one of the methods described above, the computer program product being in particular a computer-readable storage medium.

A further aspect of the present invention relates to an evaluation device for evaluating images of an analyte image sequence, comprising means for carrying out one of the methods described above.

Preferably, the evaluation device is in particular a microscope.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

The invention is explained in more detail below on the basis of the examples illustrated in the drawings. The drawings show in:

FIG. 1 schematically shows a side view of a system for use with the method for identifying analytes in an image sequence according to one embodiment;

FIG. 2 schematically shows a device for use with the method according to one embodiment;

FIG. 3 schematically shows a method for generating an image sequence by labeling analytes with markers in a plurality of coloring rounds and detecting the markers with a camera, as carried out before identifying analytes in the image sequence;

FIG. 4 schematically shows a method for providing source data for training a processing model for processing analyte image sequences;

FIG. 5 is a flow diagram showing a method for providing source data for training a processing model for processing analyte image sequences;

FIG. 6 schematically shows a method for providing source data for training a processing model for processing analyte image sequences;

FIG. 7 schematically shows a method for providing source data for training a processing model for processing analyte image sequences;

FIG. 8 schematically shows a device for providing source data for training a processing model for processing analyte image sequences;

FIG. 9 is a schematic plot of typical characteristic spectra of components of an imaging device according to one embodiment;

FIG. 10 schematically shows a device for providing source data for training a processing model for processing analyte image sequences according to one embodiment.

### DETAILED DESCRIPTION OF THE EMBODIMENTS

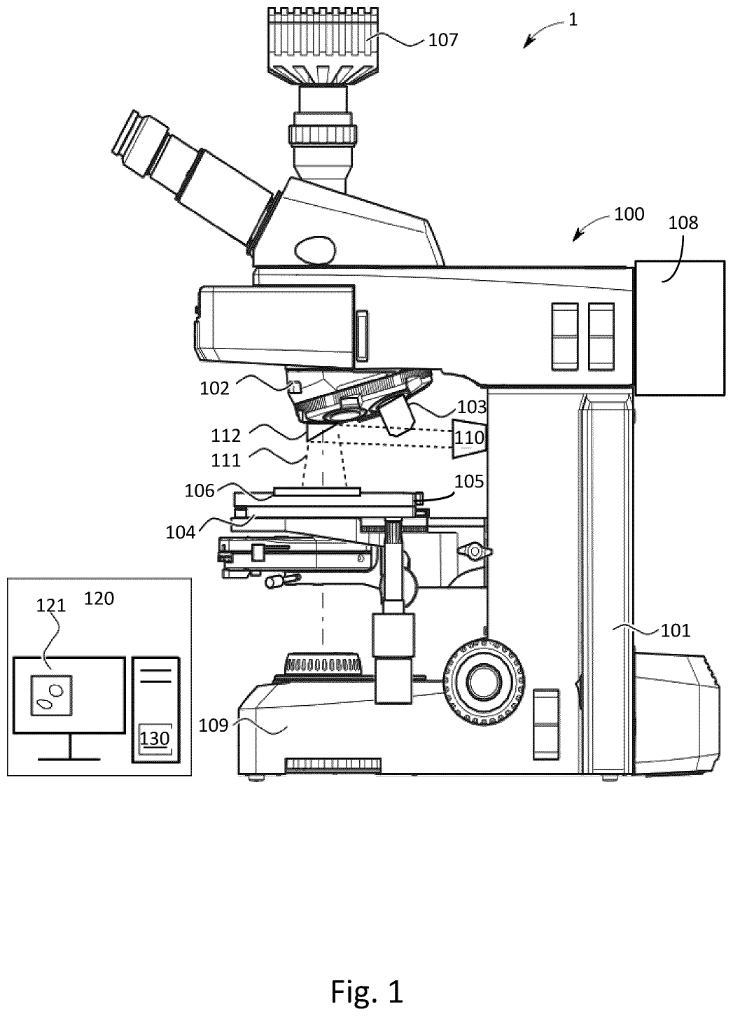

An exemplary embodiment of a source data acquisition system 1 comprises a microscope 100, a control device 120 and an evaluation device 130. The microscope 100 is communicatively coupled to the evaluation device 130 (for example to a wired or wireless communication link). The evaluation device 130 can evaluate microscope images 220 captured with the microscope 100 (FIG. 1). If the source data acquisition system 1 comprises a machine learning model, for example a neural network, it is also referred to as a machine learning system.

According to the embodiment illustrated, the microscope 100 is a light microscope. The microscope 100 comprises a stand 101 which comprises further microscope components. The further microscope components are in particular an objective changer or objective turret 102 with a mounted objective 103, a sample stage 104 with a holding frame 105 for holding a sample carrier 106 and a microscope camera 107.

If a sample is clamped into the sample carrier 106 and the objective 103 is pivoted into the microscope optical path, a fluorescence illumination device 108 can illuminate the sample for fluorescence recordings, the microscope camera 107 can receive the fluorescence light as detection light from the clamped sample and can record a microscope image 220 in a fluorescence contrast, If the microscope 100 is to be used for transmitted light microscopy, a transmitted light illumination device 109 can be used in order to illuminate the sample. The microscope camera 107 receives the detection light after passing through the clamped sample and records a microscope image 220. Samples can be any objects, fluids or structures.

The microscope 100 optionally comprises an overview camera 110 with which overview images of a sample environment can be recorded. The overview images show the sample carrier 106, for example. A field of view 111 of the overview camera 110 is larger than a field of view during a recording of a microscope image 220 with the microscope camera 107. The overview camera 110 looks at the sample carrier 106 by means of a mirror 112. The mirror 112 is arranged on the objective turret 102 and can be selected instead of the objective 103.

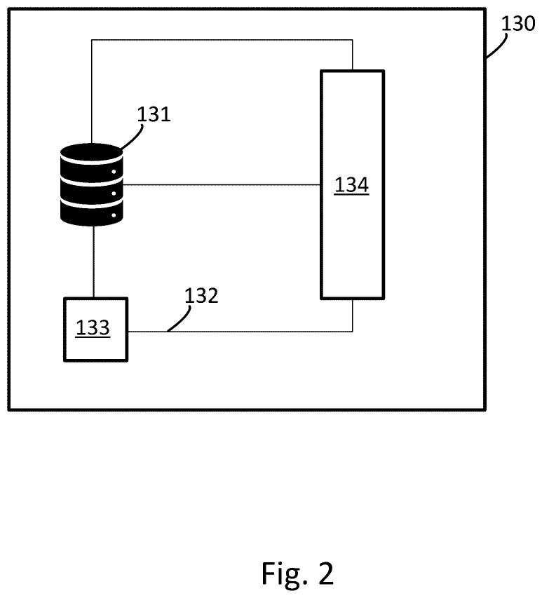

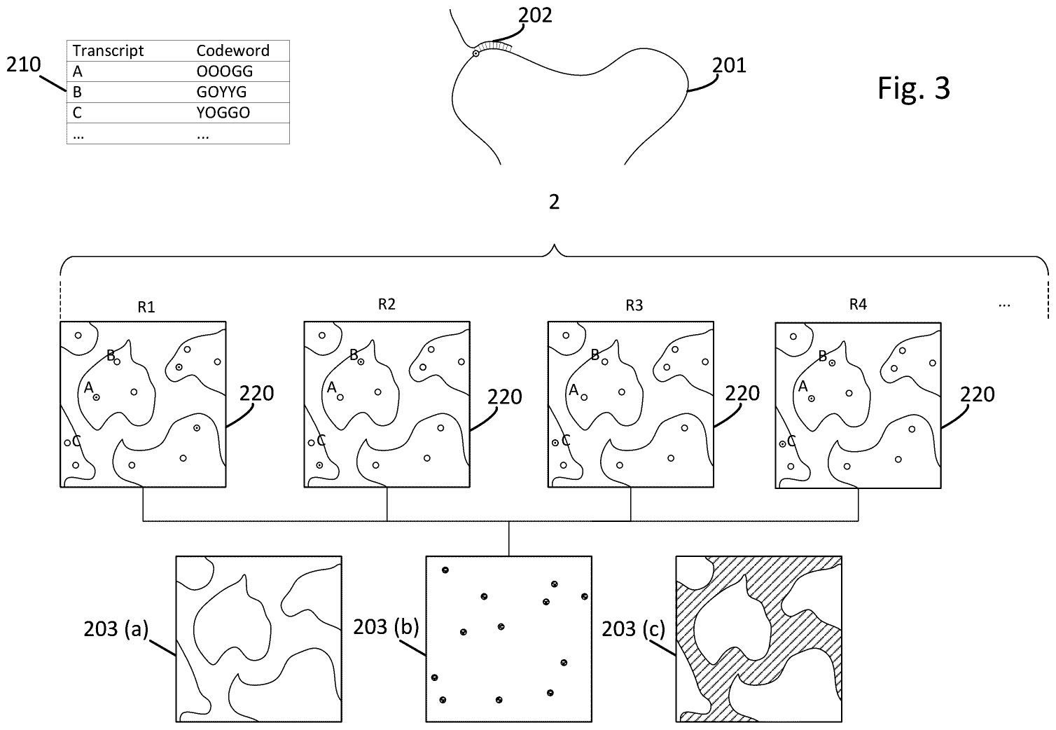

According to this embodiment, the control device 120, as illustrated schematically in FIG. 1, comprises a screen 121 and the evaluation device 130. The control device 120 is configured to control the microscope 100 to record image sequences 2 (see FIG. 3) of microscope images 220, in particular to record analyte image sequences 2, and to store the image sequences 2 recorded by the microscope camera 107 on a memory module 131 (see FIG. 2) of the evaluation device 130. The recorded microscope images 220 can be displayed on the screen 121 if required. The evaluation device 130 is configured to process the recorded microscope images 220.

The evaluation device 130 comprises various modules which exchange data via channels 132. The channels 132 are logical data connections between the individual modules. The modules can be designed both as software modules and as hardware modules.

The evaluation device 130 comprises the memory module 131. The memory module 131 stores the microscope images 220 recorded by the microscope 100 and manages the data to be evaluated in the evaluation device 130.

The evaluation device 130 comprises the memory module 131, by means of which image data of the image sequence 2 are provided and stored. A control module 133 reads image data of the image sequence 2 and a code book 210 from the memory module 131 and forwards the image data and the code book 210 to an analysis module 134. According to one embodiment, the control module 133 reads a microscope image 220 and inputs it into the analysis module 134.

According to some embodiments of the present invention, the source data acquisition system 1 is designed to provide source data for training a processing model for processing analyte image sequences 2. According to some embodiments of the present invention, an analyte image sequence 2 is an image sequence recorded by labeling analytes with markers in a plurality of coloring rounds and detecting the markers with the source data acquisition system 1.

The analysis module 134 is designed to process microscope images 220 which are recorded for compiling the source data.

In particular, the analysis module 134 can comprise a processing model, in particular a machine learning model. In particular, the machine learning model is implemented as a neural network. During the training of the machine learning model, the control module 133 controls the analysis module 134 such that the analysis module 134 reads some of the data of an annotated data set from the memory module 131 and inputs it into the machine learning model. The analysis module 134 determines an objective function on the basis of the output data of the machine learning model and on the basis of target data of the annotated data set and optimizes the objective function by adapting the model parameters of the machine learning model on the basis of the objective function.

In particular, the optimization of the objective function is carried out by means of a stochastic gradient descent method. In the stochastic gradient descent method, only a small subset, referred to as batch, of the training data of the annotated data set is used in each case. The control module 133 determines, for each input datum of the batch on the basis of the output datum output by the machine learning model and the target datum of the annotated data set corresponding to the input datum, an objective function which captures a difference between the output datum and the target datum. The control module 133 then calculates a gradient for each of the calculated objective functions with respect to the model parameters of the machine learning model, sums the calculated gradients over the batch and determines the mean value. From the mean value, the control module 133 determines updated model parameters for the machine learning model by so-called back propagation. The machine learning model is newly initiated by the control module 133 with the updated model parameters in the analysis module 134 and a next step of the stochastic gradient descent method is carried out.

The training of the machine learning model is terminated as soon as it is achieved by the optimization of the objective function that the objective function reaches a predetermined threshold value.

If the training has been completed, the control module 133 stores the most recently used model parameters of the machine learning model in the memory module 131, in particular together with context information, such that the machine learning model just trained can be identified again later and can be initialized, for example, for a further training or the inference.

As an alternative to the stochastic gradient descent method, other methods can also be used. In particular, any other training method can be used.

In the inference, the control module 133 initiates a fully trained machine learning model in the analysis module 134. After the initiation of the fully trained machine learning model, the analysis module 134 is configured for processing new image data of an analyte image sequence. During the processing, the control module 133 reads the image data from the memory module 131 and forwards them to the analysis module 134; the analysis module 134 processes the inputted data. The output data output by the analysis module 134 are in turn stored in the memory module 131 and can be displayed on the screen 121, for example.

Quite generally, source data comprise the data of a physical system which are necessary for preparing an annotated dataset. According to some embodiments of the present invention, source data here each comprise data of an analyte image sequence 2. According to the present embodiment, the source data acquisition system 1 records the source data, here in particular the analyte image sequence 2. Depending on which type of processing model is to be trained with the annotated data set, input data and target data are selected from the source data such that a corresponding processing model can be trained by means of the annotated data set to execute a desired processing mapping.

In the sense of the present invention, analyte image sequences 2 are image sequences in which analytes 201 in a sample are labeled with markers 202 in a plurality of coloring rounds according to a codebook 210 such that a signal sequence comprising colored signals and uncolored signals results over the analyte image sequences 2 for image regions that capture image signals of analytes 201, wherein the markers are selected according to the codebook 210 such that a sequence of colored signals and uncolored signals results for the signal sequence of a specific analyte type corresponding to an order of markers to be coupled to the analyte type in the respective coloring rounds stored in the codebook 210 for the analyte type.

The coupling of the markers 202 to the analytes 201 can be carried out either individually for all markers and coloring rounds, or markers 202 with n different fluorescent dyes are coupled to the analytes 201 in the sample in a marking round. For each of the n fluorescent dyes coupled to analytes 201 in a marking round by means of the markers 202, a microscope image 220 is recorded in a corresponding color channel. Each of the n recorded microscope images 220 then corresponds to one of the n coloring rounds of a marking round. Alternatively, a number of markers to be coupled can also be determined individually or predefined in each marking round, in particular by the codebook 210.

After the recording of the n microscope images 220, the markers 202 are again decoupled from the analytes 201. After the markers 202 have been again decoupled from the analytes 201, the analytes 201 can again be labeled with markers 202 with n different fluorescent dyes in a new marking round, wherein the respective fluorescent dye of the marker 202 to be coupled to the respective analyte 201 is selected according to the codebook 210 for the respective marking round or coloring round. Some of the analytes 201 to be identified can also not be labeled at all with a marker 202 in individual ones of the different marking rounds.

Since an order in which the fluorescent dyes couple to the analytes of the different analyte types is selected according to a specification in the codebook, the signal sequences of analytes resulting therefrom can be assigned to an analyte type according to the specification in the codebook after all coloring rounds have been recorded. The signal sequence to be expected for a specific analyte type is also referred to as a target signal sequence.

According to an alternative, initially only a single microscope image 220 can also be carried out per marking round by means of a fluorescence recording with a broad fluorescence excitation spectrum which simultaneously excites the fluorescence of all n fluorescent dyes used in the marking round. The recorded microscope image 220 is then converted into the respective n fluorescence contrasts after the recording by means of suitable filters, in particular software filters, so that the n microscope images 220 corresponding to the n coloring rounds of a marking round are again provided.

According to this embodiment, the codebook 210 comprises target bit sequences, wherein a true value is assigned to each expected colored signal and a false value is assigned to each expected uncolored signal.

According to a further embodiment, only markers with a single fluorescent dye are used per marking round. For this case, the dyeing steps are precisely equal to the marking rounds.

According to one embodiment, the codebook 210 is defined before the first labeling of analytes with markers. According to an alternative, the codebook 210 is defined in the course of the different coloring rounds, for example, based on the previously recorded coloring rounds.

If only a specific processing model for executing exactly one specific processing mapping is to be trained on the basis of the source data, the source data can be stored directly as annotated data set with input data and target data. If, instead, different processing models for executing different processing mappings are to be trained at a later time on the basis of the source data, then all the required data are stored in the source data. During training of the respective processing model, the annotated dataset can then be generated in each case from the source data, or the input data and the target data can be determined or correspondingly read out.

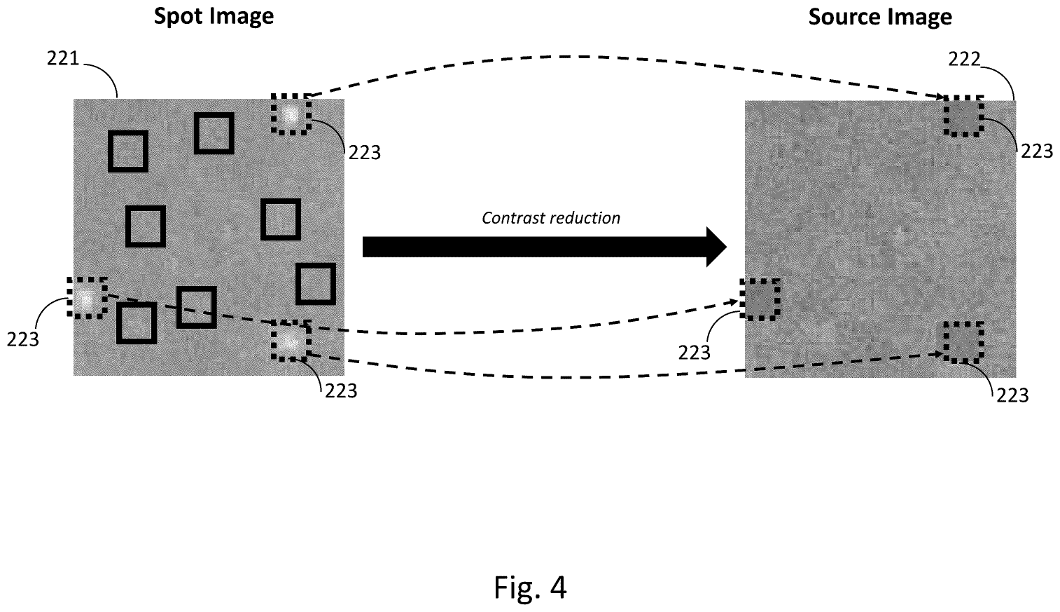

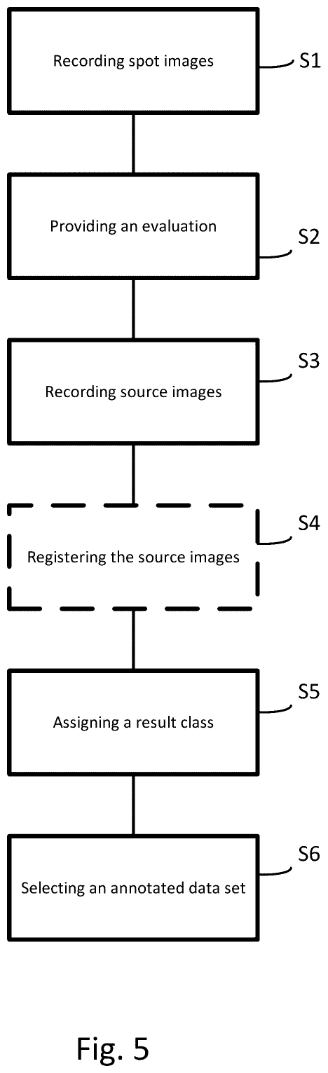

A method for providing source data in particular using the above-described source data acquisition system 1 is described below with reference to FIGS. 3-5.

In the described method for providing source data, in step S1 initially n spot images 221 of a marking round of a subarea of the sample are recorded.

The recording of the n spot images 221 initially comprises selecting the subarea of the sample in which the spot images 221 are recorded. According to this embodiment, the sample is a microtitre plate and the n spot images 221 are preferably recorded in an edge region of the microtitre plate.

In the case of microtitre plates above a certain size, drying out effects occur in the edge region, which is why parts of a sample in the edge region can no longer be examined in the later course of an experiment or an analysis of the sample on account of the drying out. Therefore, the spot images 221 are preferably recorded in the edge region of the sample, also referred to as sacrificial subarea of the sample.

To record the n spot images 221, the analytes 201 in the sample are labeled at least in the sacrificial subarea with the n markers of the first marking round according to the specifications of the codebook 210 and the n spot images 221 are recorded. For this purpose, initially a color image is either recorded which is then decomposed into the different fluorescence contrasts in accordance with the colors of the markers, in particular by means of software filters, or a separate image is recorded as a spot image for each fluorescence contrast. If, for example, markers 202 with n different fluorescent dyes are used in a marking round, n spot images 221 are accordingly also recorded. Typically, n=1, 2, 3 or else 4, but the number n of different fluorescent dyes used can also be greater.

Preferably, the number of fluorescent dyes is in particular 2, 3 or 4.

In step S1, the microscope camera 107 preferably initially records the n spot images 221 only at one location in the sacrificial subarea of the sample. After the recording of the n spot images 221 at the specific location in the sample, the microscope images are forwarded to the analysis module 134 for further processing.

In step S2, an evaluation of the image regions based on the image signals is provided and from the evaluation, spot image regions 223 of the n spot images 221 are identified in the n spot images 221 of the current marking round.

The examinations of analyte image sequences 2 have shown that a part of the analytes 201 agglomerates is in the sample. This means that in small subareas, so-called agglomeration subareas, of the sample, a large number of an analyte 201 of a certain analyte type in each case occur in an accumulated manner. Since the agglomerating analytes 201 are coupled to the markers 202 of the respective coloring round during the recording of the spot images 221, the agglomerating analytes 201 which should supply a colored signal in the respective coloring round according to the codebook 210 appear as particularly bright spots in the spot images 221. These bright spots can often be identified with the naked eye in the spot images 221.

According to some embodiments of the present invention, after the recording of spot images 221, an evaluation of the spot images 221 is provided. According to this embodiment, the image regions of the spot images 221 are identified as spot image regions 223 whose image signals (of colored signals) lie above a spot threshold value.

According to one embodiment, the analysis module 134 comprises a classic comparison operator, with which image signals of image regions of a microscope image 220, here the spot images 221, can be compared with a threshold value, the spot threshold value. The analysis module 134 receives a microscope image 220 and processes the microscope image 220 in accordance with a respective method to be carried out. After the processing of the microscope image 220, the analysis module 134 returns the microscope image 220 to the memory module 131 again for storing the processed microscope image 220.

According to the present embodiment, the spot threshold value is a threshold value defined before the recording of the analyte image sequence 2.

According to an alternative, the spot threshold value can be determined individually for each spot image 221 based on all image signals of a spot image 221. For example, the spot threshold value is a plurality of standard deviations above an average value of the image signals of the respective, entire spot image 221.

According to an alternative, the spot threshold value can be determined individually for each image region of a spot image 221 based on the local image signals around an image region of a spot image 221. For example, the spot threshold value is a plurality of standard deviations above an average value of the image signals of the surrounding of the respective image region of the spot image 221.

According to a further alternative, the evaluation can also be carried out by means of a simple classic spot or blob detection. In computer vision, blob detection methods aim at identifying regions in a digital image which differ in their properties, such as brightness or color, from the surrounding regions. Informally, a blob is a region of an image in which some properties are constant or approximately constant; all points in a blob can be considered to be similar to one another in a certain sense. The most common method for blob recognition is convolution.

As an alternative to the blob detection, other classic image processing methods can also be used for feature acquisition in images, in particular a Viola Jones detector or a histogram of oriented gradient detector, HoG detector.

As a further alternative, however, a semi-automatic method for evaluating the image regions can also be used for identifying the spot image region. For this purpose, a user can, for example, mark an image point of a spot image region, whereupon, for example, a blob is determined for the surrounding image points or one of the other above-mentioned classic image processing methods can be used for identifying the spot image region.

As a further alternative, a structure-based metric can also be used to provide the evaluation, wherein the structure-based metric evaluates the spot image region with a spot evaluation in particular based on a morphology of an object, i.e. here the spot image regions.

According to an alternative, a machine learning model can also be implemented in the analysis module 134. For example, the machine learning model can be implemented as a neural network, the neural network is then trained, for example, to identify the particularly bright image regions in microscope images 220, here the spot images 221, and is also referred to as a candidate identification model.

In particular, the candidate identification model can be designed to define the spot threshold value based on the image signals of the respective microscope image 220.

In particular, the candidate identification model can be designed to identify image regions as spot image regions 223 if their image signal lies above the spot threshold value defined by the candidate identification model.

In particular, the candidate identification model can be designed to also output the spot threshold value or else the candidate identification model does not output a spot threshold value, but all image regions identified as spot image regions 223 have an image signal above a spot threshold value. That is to say, although the candidate identification model identifies all image regions whose image signals lie above a spot threshold value, the candidate identification model does not necessarily explicitly determine the spot threshold value, but rather this results based on the image region of the spot image identified as a spot image region, wherein a lowest image signal of the image signals of the spot image regions is just greater than the spot threshold value.

The spot threshold value can furthermore also be determined individually for each image region based on image signals of image regions surrounding the image region to be evaluated. Alternatively, the candidate identification model outputs an evaluation for each image region, wherein the evaluation comprises in particular a spot evaluation and a non-spot evaluation.

For example, the candidate identification model can be a classification model which assigns one of, for example, two classes to each image region of a spot image 221, wherein the at least two classes comprise the classes of “spot image region” and “no-spot image region”.

Alternatively, however, the learned candidate identification model can also be implemented as an evaluation model which outputs an evaluation value for each image region; for all spot image regions, the evaluation value lies above the spot threshold value. The spot threshold value can in turn be defined beforehand or be determined directly by the evaluation model. In particular, the evaluation model can output a probability of whether the respective image region is a spot image region 223.

Alternatively, however, the processing model can also output a spot threshold value for the entire image from the spot image 221. The spot threshold value is stored together with the respective spot image 221 in the memory module 131 and, in the following further processing of the spot images 221, the image regions with image signals above the stored spot threshold value are interpreted as spot image regions 223. In particular, the stored spot threshold value can be interpreted together with the spot analyte image sequence as annotation in the source data, after which all image regions in the spot analyte image sequence with an image signal above the spot threshold value are identified as spot image regions.

Alternatively, the spot threshold values can be determined for each of the spot images 221, but also respectively for the different image regions, and correspondingly stored.

According to a further alternative, a spot threshold value can be determined individually for each spot image 221. This is expedient in particular if different signal levels of the image signals are to be expected during the recordings of the different coloring rounds.

According to a further alternative, for example, for each spot image 221, a corresponding map can be output in which, for example, all spot image regions 223 are suitably marked. For example, a bitmap is output for each of the spot images 221, in which it is specified for each image region of the spot image 221 whether the respective image region is a spot image region 223 or not.

According to the present embodiment, the analysis module 134 determines the spot image regions 223 for all spot images 221 of the marking round as described and stores them in the memory module 131.

According to the embodiment, all n spot images 221 of the marking round are processed in step S2, i.e. the spot image regions 223 are identified in the respective spot images 221. The identified spot image regions 223 are then stored by means of the memory module 131.

According to one alternative, however, even after the labeling of the analytes 201 and the recording of a spot image 221, initially only this one spot image 221 can be analyzed in order to identify spot image regions 223 in the one spot image 221. If the recording of this first spot image 221 yields, for example, that a number of spot image regions 223 is too small in order to generate therefrom an annotated dataset with a sufficient number of exemplary data, the control device 120 can control the source data acquisition system 1 such that further spot images 221 are recorded at another subarea of the sample in order to verify whether a density of spot image regions 223 is sufficiently high there or whether the two subareas of the sample are then sufficient to record a sufficient amount of source data.

If a marking round comprises a plurality of spot images 221 respectively corresponding to a different fluorescence contrast, initially all the spot images 221 recorded in a marking round can also be analyzed in order to identify the spot image regions 223. If all spot image regions 223 are identified, the analysis module 134 can decide accordingly whether a sufficient number of spot image regions 223 were found in the recorded spot images 221.

Step S3 comprises recording source images 222 corresponding to the n spot images 221. The capturing of the source images 222 comprises reducing the scene contrast in order to record source images 222 registered to the spot images 221 with a scene contrast reduced compared to the spot images 221.

In particular, the scene contrast in image subareas around the spot image regions 223 of the source images 222 is reduced compared to image subareas around the spot image regions 223 of the spot images 221.

According to the first embodiment, the source images 222 are recorded after the recording of the spot images 221 such that the sample is not shifted or moved in the microscope 100, which is why the source images 222 are registered to the spot images 221 and an optional registration step can be omitted.

According to an alternative, however, the source images 222 can initially be registered to the spot images 221 with a registration step after the recording. For this purpose, for example, it is expedient initially to record a source image 222 with virtually identical scene contrast so that the spot image regions 223 of the source image 222 can be registered well to the spot image regions 223 of the spot image 221. For the registration, an iterative closest-point algorithm (ICP), for short ICP algorithm, is used, for example, with which a spot image 221 can be registered to a source image 222 on the basis of the found spot image regions 223 to one another, provided that the image signal of the spot image regions 223 in the source image 222 is still sufficiently high, in particular high enough to be able to identify the spot image region well. After a first source image 222 has been registered to the spot image 221, the further source images 222 are recorded such that they are registered to the spot image 221 and the first recorded source image 222. This is possible in particular when none of the mechanical components of the microscope 100 have to be moved during the carrying out of the bleaching and during the illumination and recording of the source images 222.

A registration step can be omitted in particular when the sample is not moved in the microscope 100 between the recordings and also the other (optical) components of the microscope 100 are not moved or otherwise adjusted. If a plurality of spot images 221 with different fluorescence contrast are recorded in a marking round, if n>1 is the number of fluorescence contrasts used per marking round, source images 222 are accordingly also recorded for each of the different fluorescence contrasts.

Are the spot-Bildbereiche of the source image 222 not more to the spot-Bildbereiche 223 of the spot image 221, so is preferably a registration step.

As a scene contrast of an image, in the photographic, a brightness ratio between a brightest and a darkest image-relevant image region of the image. According to this embodiment, in the most of the recorded spot images 221, the spot image regions 223 are the brightest image regions.

Image relevant according to some embodiments of the present invention, are image regions which comprise sample structures and in which the sample structures are depicted without imaging error. Imaging errors here can be, for example, reflections at non-sample structures or the like, in particular at the sample edge. Imaging errors can have, for example, a very high image signal without structures of the sample being reproduced there. Such imaging errors are in particular neglected in the determining of the scene contrast.

Depending on the manner in which the scene contrast is reduced, the reducing of the scene contrast can relate in particular to the reducing of the scene contrast around spot image regions 223 of a specific fluorescence contrast. For example, a microscope image 220 can simultaneously be a spot image 221 for a first fluorescence contrast and a source image 222 for a second fluorescence contrast. Accordingly, the scene contrast in these images is reduced respectively only for the subareas of the microscope image 220 which are part of the source image 222.

According to the first embodiment, the reducing of the scene contrast is carried out by photo bleaching. In fluorescence microscopy, photo bleaching refers to a process of repeated illumination of a sample, wherein the intensity of the fluorescence light emitted by the markers 202 excited for fluorescence is reduced with each illumination and the image signal of the markers 202 is thus reduced in a recorded microscope image 220 if a distance between two successive illuminations is selected to be sufficiently short.

If, on the other hand, a distance between two successive illuminations is selected to be too long, the fluorescent portions, that is to say in particular autofluorescent components of the sample and the markers 202 in the sample, regenerate and the scene contrast is not reduced or substantially not reduced.

According to the first embodiment, the image signals of background image regions surrounding the spot image regions 223 and detecting autofluorescent structures regenerate more quickly between two illuminations than the image signals of the spot image regions 223. Thus, a distance between two successive illuminations during photo bleaching can be selected precisely such that the image signals of the background image regions remain approximately the same, while the image signals of the spot image regions 223 are reduced with each illumination.

According to this embodiment, the photo bleaching is continued until at least one source image 222 in which the image signals of the spot image regions 223 lie below the spot threshold value has been recorded in the respective marking round for each of the n fluorescence contrasts of the respective marking round.

According to an alternative, instead of a spot threshold value, an evaluation of the spot image regions can be carried out respectively and the photo bleaching is carried out until the evaluation of the spot image region results in a non-spot evaluation.

According to one configuration of the first embodiment, a number of illuminations during photo bleaching can be predefined. If, for example, a bleaching behavior of a sample is well known, then after the recording of the spot image 221 it is possible to determine on the basis of the image signals in the spot image regions 223, based on the known bleaching behavior, how often the sample has to be illuminated so that the image signals in the spot image regions 223 are reduced below the spot threshold value by the illumination of the sample.

If a bleaching behavior and a brightness of the spot image regions are known, in particular, then, for example, the required number of source images 222 can be recorded directly after the recording of a spot image 221 of a coloring round and thereafter it is possible to proceed directly with the next coloring round. Evaluation of the image regions and identification of the spot image regions can also be carried out only when all images of the spot analyte image sequence and of the source analyte image sequences have been recorded.

According to one configuration, a number of illuminations in the photo bleaching can be determined on the basis of the evaluation value output by the candidate identification model.

According to one configuration, the photo bleaching can comprise recording a plurality of source images 222, wherein it is determined from the source images 222 recorded during the photo bleaching whether the image signals of the spot image regions 223 of the recorded source images 222 already lie below the spot threshold value for each of the n fluorescence contrasts of a marking round for at least one of the source images 222. If a plurality of source images 222 are recorded during the photo bleaching, these plurality of recorded source images are also called bleaching sequence. The image signals for a spot image region 223 should decrease in the course within the bleaching sequence.

According to one configuration, the source images 222 of a bleaching sequence respectively belong to the same dyeing step, in particular if the number of fluorescence contrasts in a marking round n is precisely one.

According to an alternative, in the case of photo bleaching for the case n greater than one, source images 222 can be respectively successively recorded in the n different fluorescence contrasts in the respective marking round. In particular, for example, a broadband spectrum and high intensity can be used and a source image 222 can subsequently be respectively recorded for each fluorescence contrast or color channel. Here, the source images 222 of a fluorescence contrast form a bleaching sequence.

According to one configuration of the first embodiment, a source image 222 recorded after the photo bleaching is input into a scoring model, in particular a neural network. The scoring model outputs as a result only a score on the basis of which it is decided whether the photo bleaching is continued or not. The scoring model can be, for example, a CNN (convolutional neural network), an MLP (multi-layer perceptron) or else a sequential model, e.g. a recurrent neural network (RNN). If the neural network is a CNN or an MLP, then in addition to the image signals of a currently recorded microscope image 222, a score from a preceding round is also input. If the scoring model is a sequential model, then it is sufficient, on the other hand, to respectively input a most recently recorded microscope image 222 into the scoring model.

According to the first embodiment, the image signals of the spot image regions 223 lie below the spot threshold value for at least the last recorded source image 222 and at least the last of the recorded source images 222 of each of the n fluorescence contrasts or each of the n colorant contrasts of a marking round is stored in the memory module 131. In particular, the image signals of the spot image regions 223 lie below the spot threshold value for each colorant contrast for at least one of the recorded source images 222.

According to one configuration of the first embodiment, a plurality of the source images 222 recorded during the photo bleaching which form a bleaching sequence can also be stored in the memory module 131 per colorant step. Accordingly, a plurality of source analyte image sequences can be stored based on the bleaching sequences over all coloring rounds, wherein each source analyte image sequence respectively comprises one source image 222 per colorant step.

The image signals of at least one of the recorded source images 222 are preferably only just above an average value of the image signals of the background image regions. This source image is preferably used in training in order to train a machine learning model for processing analyte signal sequences with image signals only slightly above the image signals of background image regions.

In particular, the photo bleaching can be carried out repeatedly until the image signals in the spot image regions 223 lie below the average value of the image signals of the background image regions. A last recorded source image 222 can be stored, in particular it can be marked such that it becomes clear from the marking that here the image signals of the spot image regions 223 are not suitable for use in training for processing analyte signal sequences. On the other hand, a marking can also be omitted, since it follows immediately from the image signals of the last recorded source image 222 that the image signals are not suitable for the training, since here the colored signals in the spot image regions 223 cannot be distinguished from the background noise. In particular, source image sequences in which the image signals of the spot image regions 223 lie below the average value of the image signals of the background image regions, as described further below, can be used in the training of a registration model.

According to one configuration, the number of illuminations in the photo bleaching is predefined.

According to a further configuration, a bleaching sequence with a plurality of microscope images 220 is recorded during the photo bleaching and it is respectively determined from a decrease in the image signals in the spot image regions 223 of the source images 222 recorded during the photo bleaching how many further illuminations have to be carried out until the image signals of the spot image regions 223 lie below the spot threshold value.

If all the source images 222 of the respective marking round are recorded, i.e. at least one source image 222 for which the image signals of spot image regions 223 lie below the spot threshold value is respectively recorded for all coloring rounds corresponding to the respective marking round, steps S1 to S3 are then repeated for the further marking rounds or coloring rounds until at least one source image 222 for which the image signals of spot image regions 223 lie below the spot threshold value has been respectively recorded for all marking rounds and their respective coloring rounds.

After the recording of all source images 222, the source images 222 of all coloring rounds are stored together as source analyte image sequence as source data, from which an annotated dataset can be generated later, for example.

In addition, the source data in particular also comprise identification information in order to be able to identify the spot image regions 223 in the source images 222 of the source analyte image sequence. These can be lists of the spot image regions 223 in the source images 222. Alternatively, the spot analyte image sequence can also be stored together with the source analyte image sequence in the source data. In particular, the spot analyte image sequence can then be used as identification information.

The stored source data can be provided from the memory module 131 after the storing, for example for training a machine learning model.

According to a first modification of the first embodiment, the reducing of the scene contrast comprises changing the spectral properties of an imaging device.

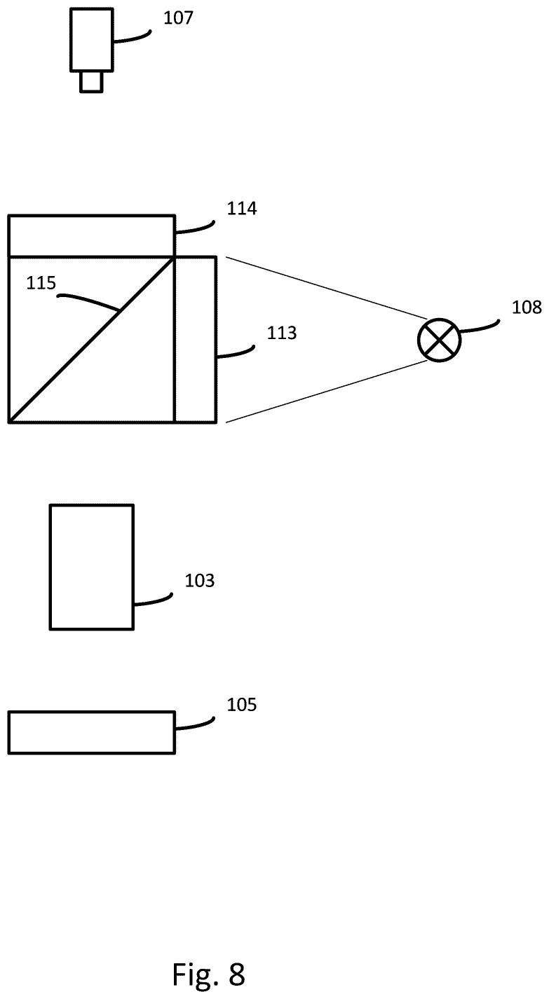

As described above with reference to the first embodiment, the imaging device comprises in particular the microscope 100 with objective 103, illumination devices 108, 109 and microscope camera 107. In addition, the microscope can also comprise different filters, for example a light source filter 113, a fluorescence filter 114, as well as a dichroic mirror 115 (see schematically FIG. 8). All of these optical components have certain spectral properties. The spectral properties include in particular a spectral transmission factor, also called a spectral transmittance, a spectral reflectivity, an illumination spectrum, an absorption spectrum and an emission spectrum. The spectral properties are detected in particular as a function of the respective wavelength in a so-called characteristic spectrum, wherein, depending on the respective component in the characteristic spectrum, for example the illumination spectrum, the spectral transmittance, the spectral reflectivity, the absorption spectrum or the emission spectrum is plotted as a function of the wavelength.

The light source filter 113 is configured to filter the illumination spectrum emitted by the light source 108, wherein the characteristic spectrum precisely comprises the spectral transmittance of the light source filter 113, which indicates which component of the incident radiation or of the incident illumination spectrum is transmitted by the light source filter 113 as a function of the wavelength.

The fluorescence filter 114 is configured to spectrally filter the fluorescence light emitted by the sample and transmitted by the dichroic mirror 115. The characteristic spectrum of the fluorescence filter again comprises the spectral transmittance of the fluorescence filter 114, which indicates which component of the incident radiation is transmitted as a function of the wavelength.

The dichroic mirror 115 is configured to reflect the light transmitted by the light source filter 113 onto the sample and to transmit the light emitted by the sample in the direction of the microscope camera 107. The spectral properties of the dichroic mirror are determined by the spectral reflectivity and the spectral transmittance; accordingly, the characteristic spectrum comprises the spectral reflectivity, which indicates which component of the radiation is reflected by the dichroic mirror as a function of the wavelength, and the spectral transmittance.

The further optical components such as, for example, the microscope camera 107 or the objective located in the beam path can have a further influence on the image signal.

Depending on the desired spectral properties, the filters 113, 114 and the dichroic mirror 115, just like the illumination device 108, also called light source 108, can be suitably selected for an imaging device according to a sample to be examined or the markers to be detected in the sample such that the sample inserted into the sample holder 105 is for example just illuminated with a spectrum which excites only certain fluorescences or certain of the markers in the sample for fluorescence.

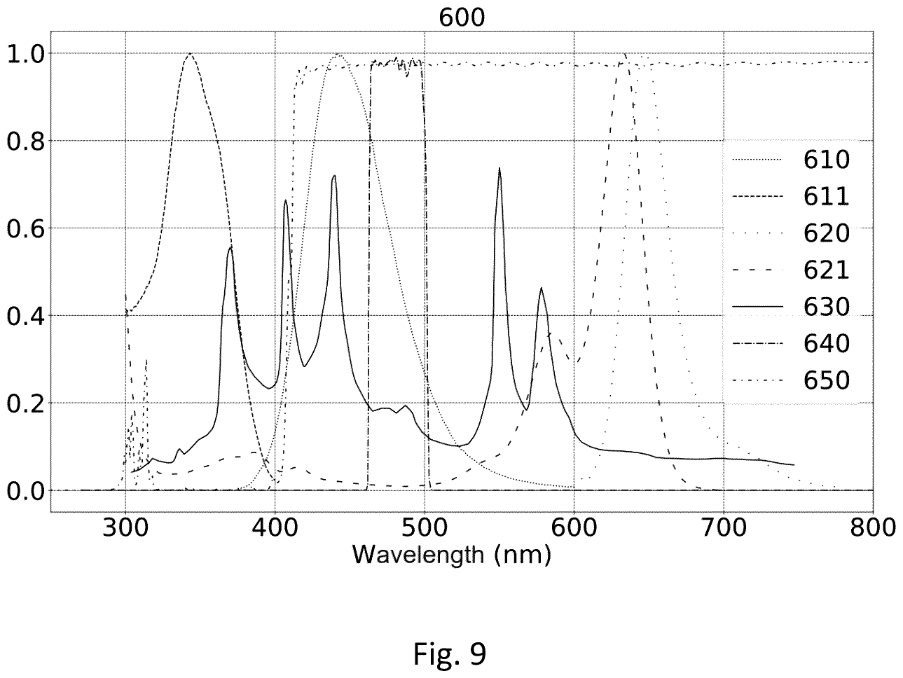

For example, FIG. 9 shows different characteristic spectra as are used in the microscopy of fluorescent samples. The wavelength is respectively selected as a unit on the abscissa, a suitable illustration is selected along the ordinate for each of the spectra, which illustration schematically represents the characteristic behavior; the person skilled in the art knows this type of schematic illustration. A first absorption spectrum 610 and a first emission spectrum 611 of a first fluorophore extend between 300 nm and 600 nm in this example. A second absorption spectrum 620 and a second emission spectrum 621 of a second fluorophore extend between approximately 400 nm and 700 nm. A first continuous spectrum 630 of a halogen metal vapor lamp is recorded here between 300 nm and 700 nm; the spectrum of the light source is also called an illumination spectrum. According to a first transmission spectrum 640, or the spectral transmittance, of a filter listed by way of example, the filter is transmissive for wavelengths in the range between approximately 460 nm and 500 nm. According to a spectral reflectivity 650 of a dichroic mirror, the mirror reflects wavelengths up to approximately 410 nm; for greater wavelengths, the dichroic mirror is transmissive.

Accordingly, light having a wavelength between 300 nm and 410 nm is reflected; this wavelength range corresponds precisely to the wavelength range in which the excitation spectrum 610 of the first fluorophore lies. An emission spectrum 611 of the first fluorophore lies above all above 410 nm; light emitted by the fluorophore in the sample therefore passes through the dichroic mirrors 115.

The first transmission spectrum 640 illustrated in the figure would only transmit a rear region of the first emission spectrum 611. For example, a spectrally transmissive region of a fluorescence filter 114 would expediently be selected such that a majority of the emission spectrum is transmitted by the fluorescence filter, that is to say for the first fluorophore approximately between 400 nm and 500 nm. Spectral filters are available with almost any bandpass filters and the illustrated first transmission spectrum 640 is listed here only by way of example and is placed in the wavelength range shown in order to show the shape of the spectrum and in order to list as few spectra as possible which overlap one another, which would disturb an overview.

According to the example shown, a spectral range of interest, as the region in which excitation and deexcitation of the fluorophores occur, extends approximately between 300 nm and 700 nm. The spectra drawn in are respectively to be understood only by way of example.

In addition to the relatively simple characteristic spectra illustrated, there are in particular still very much more complicated, for example multi-bandpass filters, with which a plurality of bands in a spectrum can be filtered out, but also multi-bandpass filters combined with dichroic mirrors and the like.

The number of different available fluorophores or fluorophores suitable for the respective experiment or the respective sample, and their respective characteristic spectra, are known to the person skilled in the art and can be looked up in different databases known to the person skilled in the art.

The same applies to the commonly available filters, dichroic mirrors, light sources and detectors and their characteristic spectra or characteristic optical properties, which can likewise be retrieved in databases.

Correspondingly, the person skilled in the art can select the respective components for his experiment in a suitable manner for the respective experiment and the markers to be used in each case. Some of the databases also make it possible to calculate the excitation efficiencies for selected configurations, comprising light sources, fluorophores in combination with filters used.

In particular on the basis of these databases, a user can select the components of the imaging device precisely such that the spectral properties during the use of a so-called source configuration the illumination of the sample takes place such that a scene contrast in the case of the source image recorded with the source configuration is reduced compared to a spot image recorded with a spot configuration precisely such that for colored signals the image signals of the spot image regions in the spot images lie above the spot threshold value and the image signals in the spot image regions in the source images lie below the spot threshold value.

In particular, the source configuration and the spot configuration relate to a coloring round and a fluorescence contrast to be recorded in the respective coloring round.

In particular, the light source used in the illumination with the source configuration and the spot configuration can be different. Each light source emits a specific illumination spectrum. Depending on the extent to which the illumination spectrum of the light source 108 coincides or coincides with an excitation spectrum of a fluorophore, the fluorophore is more or less excited to fluoresce. A fluorescence signal of the respective fluorophore recorded as an image signal in a recorded microscope image is correspondingly higher or lower.

In particular, a light source filter 113 used in the illumination with the source configuration and the spot configuration can be different. Analogously to the use of different light sources 108, instead of using the different light sources 108, a light source filter 113, which filters the illumination spectrum of the light source 108 before it strikes the sample, can be modified between the source configuration and the spot configuration such that the scene contrast during the recording with the source configuration is correspondingly reduced such that the image signal in the spot image regions lies below the spot threshold value during the recording of the source image.

Alternatively, the fluorescence filter 114 or the dichroic mirror can also be modified in accordance with the above description. In particular, the fluorescence filter 114, the light source filter 113, the dichroic mirror 115, as well as the light source 108 can be suitably modified such that, as described above, when recording the source images 222, the scene contrast is reduced or greatly reduced as described above compared to the recording of the spot images 221.

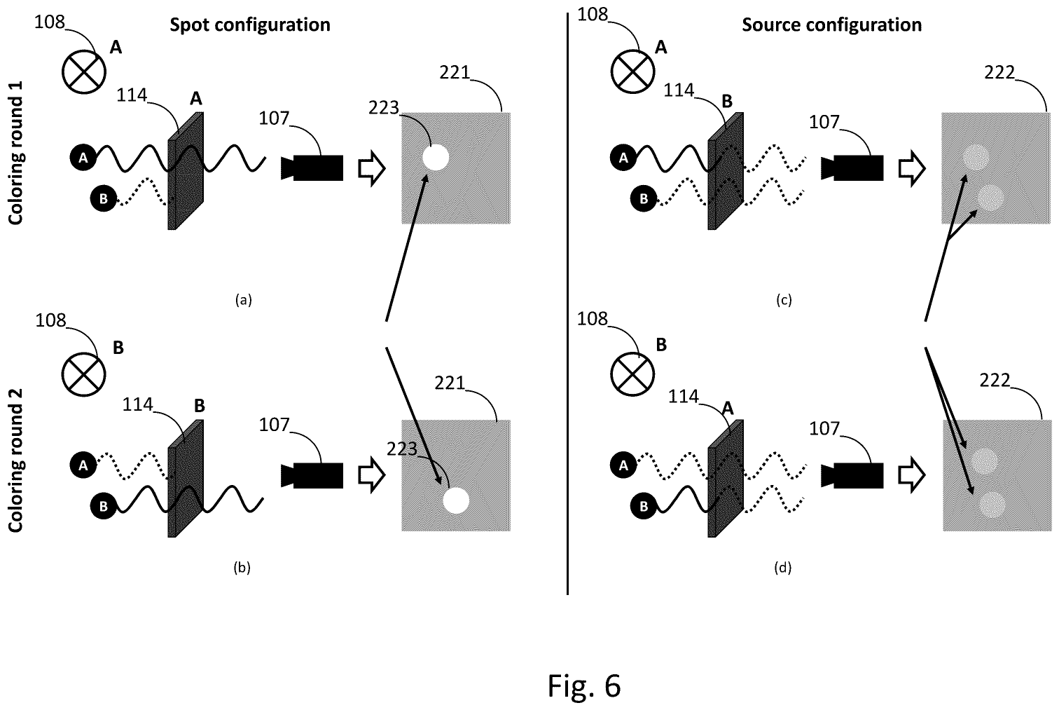

The method for providing source data for training a machine learning model is illustrated schematically on the basis of a set of spot and source configurations with a reduced degree of detail in FIG. 6. According to the illustration, a marking round comprises two coloring rounds with fluorescence contrasts A and B, or fluorophores A and B.

According to a spot configuration, light source A is used in a first coloring round of the marking round, see portion (a) of FIG. 6. The light source A has an illumination spectrum with a good match, also called overlap, with an excitation spectrum of the fluorophore A and is thus well suited to exciting the fluorescence of the fluorophore A. In addition, the illumination spectrum of the light source A has a small overlap with the excitation spectrum of the fluorophore B, for which reason a low fluorescence signal also emanates from the fluorophore B, shown in dashed lines in FIG. 6.

Furthermore, the spot configuration comprises a fluorescence filter A which is well matched to the fluorescence spectrum of the fluorophore A, i.e. the fluorescence filter A transmits the predominant proportion of the fluorescence light of the fluorophore A. In the spot image 221, the image signal of the fluorophore is shown particularly bright; the image region in which the fluorescence light of the fluorophore A is captured is correspondingly a spot image region; the image signal of a colored signal of the fluorophore A lies above the spot threshold value.

According to a spot configuration of the second coloring round of the marking round, see portion (b) of FIG. 6, a light source B is used. The light source B has an illumination spectrum with a good overlap with an excitation spectrum of the fluorophore B and is thus suited to exciting the fluorescence of the fluorophore B. In addition, the illumination spectrum of the light source B has a small overlap with the excitation spectrum of the fluorophore A, for which reason a low fluorescence signal also emanates from the fluorophore A, shown in dashed lines in portion (b) of FIG. 6. Furthermore, in the spot configuration, a fluorescence filter B is used which is well matched to the fluorescence spectrum of the fluorophore B, i.e. the fluorescence filter B transmits the predominant proportion of the fluorescence light of the fluorophore B. In the spot image 221, the image signal of the fluorophore is shown particularly bright; the image region in which the fluorescence light of the fluorophore is captured is correspondingly a spot image region, since the image signal of the fluorophore B lies above the spot threshold value.

The source configuration of the first coloring round of the marking round, see portion (c) of FIG. 6, differs from the spot configuration of the first coloring round in that the fluorescence filter B is used instead of the fluorescence filter A. According to portion (c) of FIG. 6, the fluorescence filter B filters out only a part of the fluorescence spectrum of the fluorophore A, such that an image signal of the spot image region of the fluorophore A lies below the spot threshold value.

If the distances between the excitation and emission spectra of the fluorophores A and B and the corresponding fluorescence filters are selected to be too small, a certain component of the emission spectrum of the fluorophore A can also be transmitted by the fluorescence filter B; this phenomenon is also referred to as bleed through. Bleed through is precisely not desired for many applications, since the color channels are intended to be separated cleanly. In this modification of the first embodiment, the bleed through can be selectively used during the recording of the source analyte image sequence or for reducing the scene contrast.

On account of the small distance of the excitation and emission spectra of the fluorophores A and B, the fluorescence filter B cannot separate the emission spectra of the fluorophores A and B from one another, for which reason, as shown in portion (c) of FIG. 6, a small image signal is also captured in the spot image region of the analyte marked with the fluorophore B. Accordingly, during the compilation of the source signal sequence for the analyte type marked with the fluorophore B which, according to the codebook, has an uncolored signal in the first coloring round, the uncolored signal of the spot image region does not have to be used from the microscope image recorded with the source configuration and shown in portion (c) of FIG. 6, since it is precisely the case here that the fluorophore B slightly excited by the light source A is bled through by the fluorescence filter B. Instead, for the first coloring round for the source signal sequence of the analyte type marked with the fluorophore B, it is necessary to use the image signal of the spot image region from the microscope image recorded with the spot configuration and illustrated in portion (a) of FIG. 6.

The source configuration of the second coloring round of the marking round, see portion (d) of FIG. 6, differs from the spot configuration of the second coloring round in that the fluorescence filter A is used instead of the fluorescence filter B. According to portion (d) of FIG. 6, the fluorescence filter A filters out a part of the fluorescence spectrum of the fluorophore B, such that an image signal of the spot image region of the fluorophore B lies below the spot threshold value. In the case of the source configuration of the second coloring round, the bleed through occurs again, in this configuration with respect to the fluorescence signal of the fluorophore A, which is excited to fluorescence at least to a small extent by the illumination with the light source B. According to portion (d) of FIG. 6, the fluorescence signal of the fluorophore A is transmitted by the fluorescence filter A.

As already described above with reference to portion (c) of FIG. 6, also for the analyte type marked with the fluorophore A which, according to the codebook 210, has an uncolored signal in the second coloring round, during the compilation of the source signal sequence for the second coloring round, the image signal of the spot image region from the microscope image of portion (b) of FIG. 6 recorded with the spot configuration has to be used.

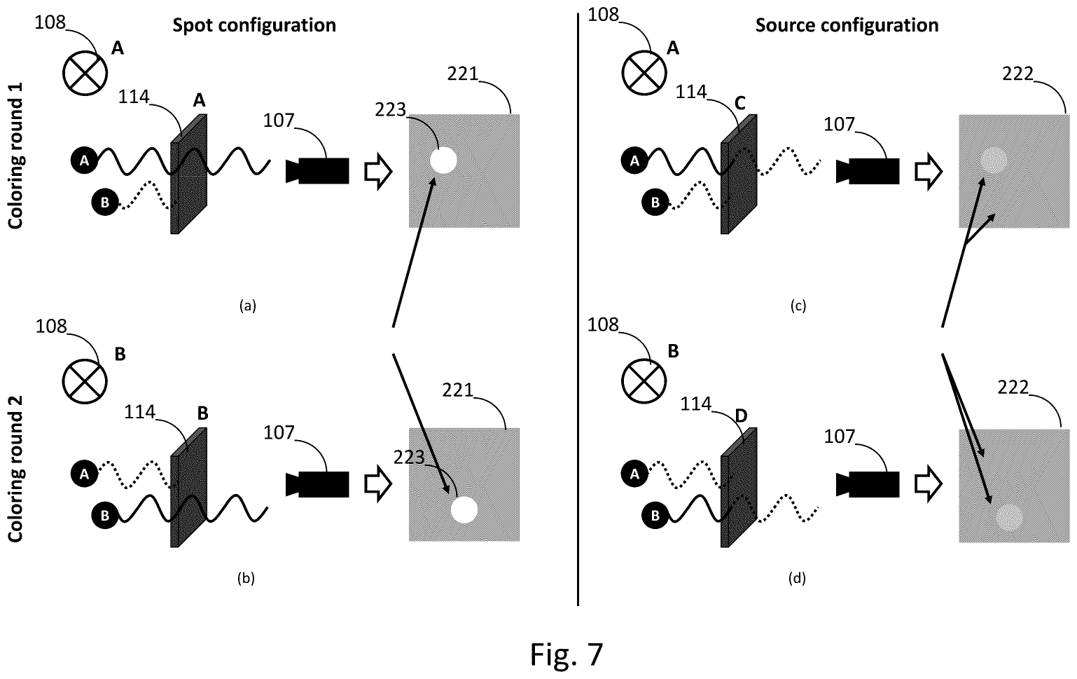

An alternative illustrated in FIG. 7 differs from the configurations of FIG. 6 in that different fluorescence filters are respectively used in the source configurations of the first and the second coloring rounds, in particular fluorescence filters C and D. As illustrated in portions (c) and (d) of FIG. 7, the fluorescence filters C and D are configured such that a bleed through of the fluorophores A or B cannot be observed in the recordings with the source configurations of the first and second coloring rounds.

According to one configuration of the first modification, the source configuration and the spot configuration can also be selected such that, for a first fluorescence contrast, a recording with the source configuration corresponds precisely to a recording of a second fluorescence contrast with the spot configuration, i.e. source images 222 recorded for the first fluorescence contrast correspond precisely to the spot images 221 of the second fluorescence contrast. If the source configuration of a first fluorescence contrast is precisely the spot configuration of the second fluorescence contrast, this is also referred to as a so-called cross configuration. This type of cross configuration can in particular also apply conversely to the spot configuration of the second fluorescence contrast and the source configuration of the first fluorescence contrast. Alternatively, however, further fluorescence contrasts for which similar cross configurations exist can also be recorded.

Alternatively, each of the coloring rounds can comprise its own source configuration and its own spot configuration, wherein the source configuration and the spot configuration are not a source configuration or a spot configuration for any other of the coloring rounds.

If the microscope 100 having the above-described cross configurations is used during the recording of the spot analyte image sequence and the source analyte image sequence, the spot image of the first fluorescence contrast, here in particular the first coloring round of a marking round, is also the source image of the second fluorescence contrast, here the second coloring round of a marking round. That is to say, the recorded microscope images are simultaneously spot images and source images, but only one of the microscope images of one of the coloring rounds of a marking round always has a colored signal with an image signal above the spot threshold value, since each analyte type is always only coupled to a specific marker of a specific fluorescence contrast. In the sense of this configuration of the first modification, a source image 222 is therefore precisely a field of view in which only spot image regions of the source signal sequence lying with reduced scene contrast lie and the scene contrast in this field of view is thus reduced at least by the spot image region of the source signal sequence.

As, when using such cross configurations, a single microscope image however comprises the colored signals respectively for two different coloring rounds, once for the spot signal sequence and once for the source signal sequence, the uncolored signals for the analyte signal sequences, in particular for the source analyte signal sequences, are missing for such analyte image sequences recorded according to the cross configuration, since the source images of one fluorescence contrast are indeed respectively the spot images of the other fluorescence contrast and therefore capture colored signals for the other fluorescence contrast which meet the condition of a spot image region and whose image signal lie above the spot threshold value. According to the codebook, however, in these coloring rounds the other fluorescence contrast as described above would have to have an uncolored signal.

Accordingly, when using such cross configurations, at least one source image having uncolored signals for the respective fluorescence contrast must also be recorded for each fluorescence contrast; these can then be used for all coloring rounds in which the respectively identified analyte type should capture an uncolored signal according to codebook 210. In order to match an image signal of the uncolored signal with the image signal of the colored signal of the respective coloring round to one another, the source contrast can be reduced with the uncolored signals for the respective fluorescence contrast in particular by means of the photo bleaching described above and the scene contrast can be reduced.

Alternatively, however, a further configuration with suitable spectral properties for recording the source image with the uncolored signals for the respective fluorescence contrast can also be recorded, for example, in particular an uncolored signal can take place, for example, by recording the sample without coupled markers, for example in autofluorescence contrast.

According to a further configuration, so-called cyclic configurations are also possible. By way of example, a cyclic configuration for n=3 different fluorescence contrasts per marking round is explained here. For this case, the cyclic configurations comprise precisely n+1=4 configurations.

A first configuration is the spot configuration of the fluorescence contrast of the first coloring round of the respective marking round. The first configuration is also selected such that image signals of spot image regions which capture fluorescence signals of the analyte types colored with the fluorescence contrast of the third coloring round can be used from the image of the first coloring round for the uncolored signals of the source-analyte signal sequences of the respective analyte types, since no bleed through of the fluorescence contrast of the third coloring round can be observed in the images of the first coloring round.

The second configuration is the spot configuration of the fluorescence contrast of the second coloring round and the source configuration of the fluorescence contrast of the first coloring round.

The third configuration is the spot configuration of the fluorescence contrast of the third coloring round and the source configuration of the fluorescence contrast of the second coloring round. In particular, the image signals of the spot image regions which capture fluorescence signals of the analyte types colored with the fluorescence contrast of the first coloring round can be used from the images of the third coloring round for the uncolored signals of the source-analyte signal sequences of the respective analyte types.

A fourth configuration is the source configuration of the fluorescence contrast of the third coloring round. In particular, the image signals of the spot image regions which capture fluorescence signals of the analyte types colored with the fluorescence contrast of the second coloring round can be used from the image recorded with the fourth configuration as uncolored signals of the source-analyte signal sequences of the respective analyte types.

In this type of cyclic configuration, n+1 different configurations are always required for n>2 different fluorescence contrasts per marking round in order to record all colored and uncolored signals required for the training for all source-analyte signal sequences.

According to the first modification of the first embodiment, a user determines the source configuration and the spot configuration.

According to a second modification of the first embodiment, the reducing of the scene contrast can comprise unequally illuminating the sample for capturing the source images. In particular, the spot subareas of the sample mapping to the spot image regions and the regions of the sample surrounding the spot subareas are just illuminated unequally such that the scene contrast is reduced.

In particular, by shorter or less intensive illumination of the spot subareas of the sample, an image signal captured in the spot subareas of the source images 222 can be considerably reduced compared to the image signal in the spot image regions of the spot images 221, since a fluorescence signal excited in the sample turns out to be considerably smaller as a result of the shorter or less intensive illumination.

For example, a microscope 100 with a laser can be used as illumination device 108. The laser is then configured to excite the spot image regions to fluorescence correspondingly shorter, in order to obtain a smaller fluorescence signal for the spot image regions.

Further configurations are explained below both for the first embodiment and for the modifications of the first embodiment.

In particular, the first embodiment and the first and the second modification of the first embodiment can be suitably combined with one another. If, for example, the optical components are not present in a laboratory for the capturing of the source analyte image sequence, a light loading for the sample can be further reduced by the combination of the first embodiment with the first modification of the first embodiment.

This takes place in particular in that the spot images 221 are recorded with a spot configuration. After the recording of a spot image 221, the image signals in the spot image regions are thereupon reduced, for example by means of the photo bleaching, and the intensity is thereupon further reduced using a source configuration, such that the image signal in the spot image regions for at least one recorded source image lies below the spot threshold value.

Equally well, after the recording of the spot image 221 using the spot configuration, it is also possible to change directly to the source configuration, and the bleaching sequence can be recorded using the source configuration.

In particular, the recording of the bleaching sequence using spot configuration and source configuration can also be combined with the selective illumination of the sample. In particular, the source configuration can also comprise information about parts of the sample to be illuminated.

In particular, the source configuration can also comprise information about a number of illuminations to be carried out during the recording of the bleaching sequence.

According to an alternative, instead of the evaluation based on the spot threshold value, other types of evaluations can also be used as described above for evaluating the image regions. The evaluation of the spot image regions for the spot configuration then results precisely in the spot evaluation and for the source images recorded with the source configuration precisely in the non-spot evaluation at least for source images of one of the source analyte image sequences.

In many of the laboratories, microscopes with a wide variety of optical components are available, in particular certain filters, light sources, dichroic mirrors can be different in each of the different laboratories. In order to keep the investment costs for further optical components as low as possible, some embodiments of the present invention provide the configuration determination model. By means of the configuration determination model, the source and the spot configuration can be determined on the basis of the optical components present, for which reason it is not necessarily required to acquire further optical components in order to be able to realize an ideal source or spot configuration for recording the source analyte image sequence and the spot analyte image sequence.

In particular, the recording of the bleaching sequence can comprise matching image signals of an autofluorescence of background image regions surrounding the spot image regions and the image signals of the spot image regions, such that the image signals of the background image regions are reduced less strongly during the recording of the bleaching sequence than the image signals of the spot image regions.

The matching of the image signals can be carried out in particular by suitably selecting a temporal distance between two successive illuminations, wherein the image signals of the autofluorescence regenerate more strongly between two successive illuminations than the image signals of the spot image regions.

Alternatively, the matching of the image signals can in particular comprise unequally illuminating the sample during the photo bleaching, in particular only the spot subareas of the sample are illuminated, i.e. the subareas of the sample captured by the spot image regions.

According to a further alternative, the matching of the image signals can also comprise suitably processing the image signals of the background image regions, wherein the image signals of the background image regions are processed such that the image signals in the background image regions remain approximately constant over all illuminations during the recording of the bleaching sequence.

In particular, the matching of the image signals can also comprise suitably selecting a concentration of the fluorescence suppressor, such that the image signals of colored signals of spot image regions approach the image signals of the background image regions.

According to one embodiment, the spot configuration and the source configuration can be determined by means of a configuration determination model based in particular on the markers used for labeling the analytes and the available microscope components.

In particular, the matching of the image signals can be carried out by means of a laser as light source, wherein the laser can also be designed such that it photo bleaches the spot image regions by intensive illumination, and a recording of the source images is carried out in particular after the photo bleaching with the laser.

According to one configuration of the first modification of the first embodiment, a configuration determination model can be used for determining the source configuration and the spot configuration.

The configuration determination model can comprise, as input data, the different markers used for labeling the analytes and outputs, as output data, configuration of the microscope to be used. In particular, the output data comprise one or more items of information about the following: a light source used, a fluorescence filter used, a dichroic mirror used, a light source light filter used. In particular, the input data comprise the optical components of the microscope available for recording the analyte image sequence.

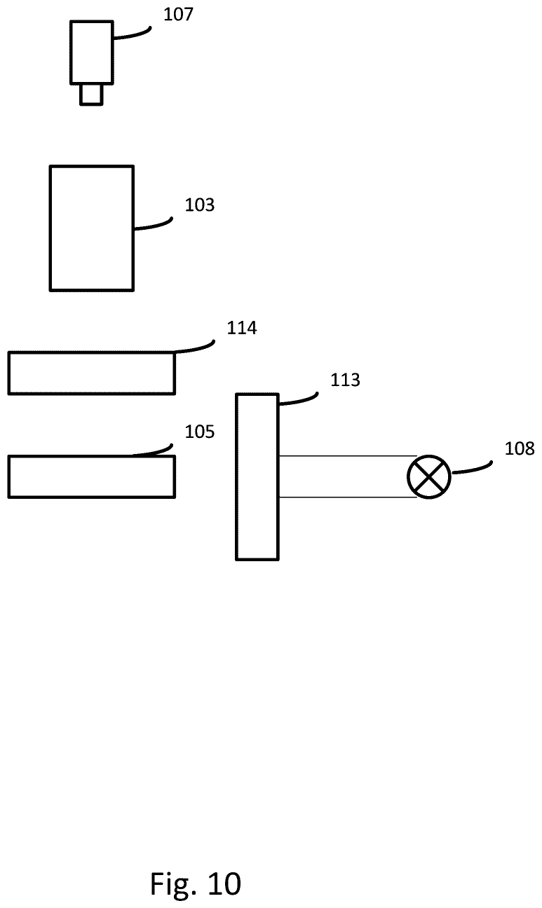

According to a modification of the first embodiment, a source data acquisition system 1 can also have an alternative construction without dichroic mirror 115, as illustrated schematically in FIG. 10. Furthermore, for example, a fluorescence filter 114 or a light source filter 113 can also be omitted.

According to a second embodiment, the present invention provides a method for providing an annotated data set. The method for providing an annotated dataset follows the method for providing source data according to the first embodiment, or can comprise the method for providing source data according to the first embodiment. Alternatively, the method can also be applied on the basis of a set of stored source data.

According to the second embodiment, the annotated dataset is provided for training a machine learning model. The machine learning model is trained with the annotated dataset in particular for processing analyte image sequences, wherein, according to the second embodiment, the processing comprises an assignment of signal sequences of the analyte image sequence to a result class, the machine learning model thus being a result class assignment model.

In step S4, initially the source images 222 of the source analyte image sequence and, provided that the source data also comprise the spot analyte image sequence, also the spot images 221 of the spot analyte image sequence of the different coloring rounds are registered to one another. The registration can be carried out by means of a classic registration algorithm, in particular an iterative nearest point method, or with a registration model trained for this purpose.

After the registration of the source images 222 of the source analyte image sequence to one another and possibly of the spot images 221 of the spot analyte image sequence, the image sequences with the images registered to one another are stored and can be further processed accordingly in step S5.

According to step S5, a result class is assigned to the signal sequences of spot image regions 223 stored in the source data on the basis of the codebook 210, or a result class is determined for the signal sequences on the basis of the codebook 210. For this purpose, initially the signal sequences of the spot image regions 223 of the spot analyte image sequence are read out. For each spot image region 223, for a coloring round in which an analyte of an agglomeration subarea in the sample mapping to the spot image region 223 is marked with a marker 202, a colored signal results which for spot image regions 223 is just equivalent to an image signal above the spot threshold value or equivalent to a spot evaluation, and for coloring rounds in which the analyte mapping to the respective spot image region 223 is not marked or coupled to a marker 202, an uncolored signal results, the image signal in this case lies significantly below the spot threshold value, it is approximately equal to an image signal of a background image region surrounding the spot image region 223.

The signal sequence of colored and uncolored signals of a spot image region 223 over the spot analyte image sequence, also referred to as spot signal sequence, are subsequently correspondingly binarized, for example the binary value “1” is assigned to the colored signals and the binary value “0” is assigned to the uncolored signals. The binarized spot signal sequences are then compared with the codebook 210 in order to assign the result class to the spot signal sequence. The result classes comprise precisely the different analyte types of the codebook 210, and a background class for signal sequences which cannot be assigned uniquely to an analyte type or for which an analyte type cannot be determined uniquely.

Spot signal sequences to which an analyte type is assigned as a result class are also referred to as spot analyte signal sequences. Spot signal sequences to which the background class is assigned as a result class are also referred to as spot background signal sequences. Since the signal sequences of the source image sequences, also called source signal sequences, are always based on signals of the same analytes as those of the spot signal sequences, the same result class can be assigned to the source signal sequences corresponding to the respective spot signal sequences. Correspondingly, source signal sequences to which an analyte type has been assigned as a result class are also referred to as source-analyte signal sequences, and the source signal sequences to which the background class has been assigned are referred to as source-background signal sequences.

According to some embodiments of the present invention, a codebook 210 comprises for each analyte type a target bit sequence, which indicates in which of the coloring rounds an analyte of the respective analyte type is labeled with a marker 202 of the respective coloring round.

According to an alternative, instead of the target bit sequence, the codebook 210 can also comprise a code word, which code word is encoded, for example, in the contrast colors, also referred to as fluorescence contrast. The target bit sequence can be determined in a simple manner from the code word. For this purpose, a bit sequence is correspondingly assigned to each encoded contrast color. If, for example, three different fluorescence contrasts are used, here for example orange, O, yellow, G and blue, B, and the different fluorescence contrasts are respectively recorded in the order orange, yellow, blue in a marking round, the bit sequence for the fluorescence contrast orange is precisely equal to “100”, for yellow precisely equal to “010” and for blue precisely equal to “001”. Corresponding bit sequences can be correspondingly selected for a different number of fluorescence contrasts.

If, in particular, the plurality of coloring rounds of a marking round are recorded in an image of the microscope 100 and the spot or source images 221, 222 are determined from the image, for example by software filters as described above, for the different fluorescence contrasts or coloring rounds, the order orange, yellow, blue can accordingly be selected as desired, but only the orders of the fluorescence contrasts of the respective coloring rounds used in the images of the analyte image sequence must coincide with the fluorescence contrasts used in the codebook for the respective coloring rounds.

In the case of multiomy, the target bit sequences are typically selected such that the analyte type can also still be reliably assigned if, for example, an incorrect signal occurs in one or two of the coloring rounds. Correspondingly, in the assigning of the analyte types the analyte type can respectively be determined as analyte type of the respective signal sequence whose target bit sequence has a best match with the binarized spot signal sequence of the spot image region.

If no unique assignment to an analyte type is possible for one of the spot signal sequences, the background class is assigned to the signal sequence as a result class; in addition, the spot image region corresponding to the spot signal sequence can be identified as background image regions.

The assignment of the spot image regions or of the spot signal sequences to the respective result class is stored as assignment information by means of the memory module 131, that is to say information about the assigned result class.

According to a step S6, an annotated dataset for training a machine learning model is selected from the stored data from the source data together with the registration information and/or assignment information stored according to step S4 and step S5 in step S6.

According to the second embodiment, the machine learning model to be trained is the result class assignment model which is trained to assign a result class to an input signal sequence of an image region. As described above, the possible result classes are in particular the different analyte types occurring in a sample, or the analyte types encoded in the codebook; furthermore, the result classes comprise at least one background class.

In the training, signal sequences of spot image regions over the source-analyte image sequences, also referred to as source-analyte signal sequence, are used as training input data. The target output contained in the annotated dataset, in particular a form of the target output, depends on an implementation selected for the result class assignment model. In principle, the target outputs contained in the annotated data set indicate the result class assigned to the respective source-analyte signal sequence of the input data.

In particular, the result class assignment model can be implemented as a neural network, in particular as a convolutional neural network (CNN), multi-layer perceptron (MLP), or as a sequential network, for example a recurrent neural network or a transformer network.

If the model is implemented as a sequential network, the signal sequences are not input into the model as a whole, but rather the image signals of the signal sequences are input individually into the model.

If the model is a convolutional network and is implemented as a sequential network, then the model first sees the image of a first coloring round, then the image of a second coloring round and then step by step the images of the following coloring rounds. In a coloring round N, the model receives only the image from the round N and has an internal state which encodes or stores the images from the rounds 1 to N−1 internally. In round N, the model then processes the internal state with the image from the coloring round N.

According to this embodiment, the result class assignment model is trained as a classification model, also referred to as classifier.

In this case, source signal sequences are assigned to a result class on the basis of a specific order of colored signals and uncolored signals on the basis of a codebook 210.

While it is often already possible with the naked eye in the case of the spot signal sequences to distinguish colored signals from uncolored signals of a spot signal sequence, a majority of the signal sequences capturing signals of analytes are based on non-agglomerating analytes present in a sample, the signal sequences of which can be distinguished analytically only with difficulty from signal sequences of the background. It is correspondingly difficult to identify the colored and uncolored signals analytically. The inventors have recognized that the signal sequences of the non-agglomerating analytes respectively have characteristic signatures depending on the respective analyte type and thus depending on the order of colored and uncolored signals based on the codebook. The characteristic signatures can comprise, for example, a specific ratio of colored to uncolored signal, colored to colored signal or uncolored to uncolored signal, on the basis of which colored and uncolored signals can be identified in a source signal sequence.

The determined ratio can be a specific distance or a difference between the image signals, a quotient of the image signals, a specific number of image signals with a higher image signal than the remaining ones, wherein the ratio can respectively be learned for a normalized image signal or for a non-normalized image signal.

The classification model is now trained by means of the suitably compiled annotated data set to identify these characteristic signatures for each analyte type and to assign a corresponding source signal sequence to the result class of this analyte type.

The classifier can be designed, for example, to output a hard assignment or a soft assignment. A hard assignment means that a result class is output directly. A soft assignment means that the classifier outputs a probability distribution over the possible result classes.

Correspondingly, the target output in the annotated dataset for each source signal sequence comprises in particular the result class corresponding to the source signal sequence, or a probability distribution, with a probability of one in the assigned result class.

Alternatively, the output can also be a vector in which each entry corresponds to a class and the result class then has the entry one.

Any of the non-spot image regions of the source analyte image sequence can be used as further input data, although no result class has been assigned to these non-spot image regions in step S5, these are assigned automatically to the background class, for example.

An automatic assignment of the non-spot image regions to the background class can be carried out in particular whenever the sample has been used during the photo bleaching respectively for an entire subarea of the sample captured by a source image during the photo bleaching. A confusion with analyte signal sequences of non-spot image regions, i.e. analyte signal sequences which are based on image signals of non-agglomerating analytes, is not to be expected since the non-spot image regions have also been used in step S2, for which reason the image signals of the colored signals of analytes in the non-spot image regions are likewise considerably reduced.

Alternatively or additionally, the annotated data set can also comprise signal sequences which are compiled randomly in order to train the identification of the background class.

According to an alternative, for example, for the case in which only the spot image regions 223 have been subjected to photo bleaching, for example with a specific illumination in particular using a laser scanner, only randomly compiled signal sequences can be used for training the background class. If, specifically, instead of the randomly selected signal sequences, signal sequences of non-spot image regions over the source analyte image sequence were used in the training, then it would be possible to randomly select a signal sequence which captures the signals of a non-agglomerating analyte, the image region of which, however, has just not been used according to this alternative. The machine learning model would thus be trained to incorrectly assign an analyte signal sequence to the background, which should be avoided, since an analyte has in fact just now been detected in the image region and the identification thereof should just now be trained by means of the method.

In addition, source-background signal sequences can also be used for training the machine learning model, so that the machine learning model also learns to distinguish the source-background signal sequences from the source-analyte signal sequences.

During the selection of signal sequences for the annotated data set from the source data, it is furthermore ensured that a sufficient number of examples is transferred into the annotated data set for each of the analyte types, so that the result class assignment model recognizes all possible result classes, in particular all analyte types, well after completion of the training.

According to one configuration, it can occur in samples that certain analyte types do not agglomerate in the sample, or that only a few of the analytes of the certain analyte types agglomerate and therefore only a few source-analyte signal sequences are present in the source data for this analyte type. In order to also provide sufficiently many signal sequences for the training of the machine learning model in the annotated dataset for these analyte types which agglomerate too rarely, it is possible, on the basis of the target bit sequence of the codebook 210 for these analyte types which agglomerate rarely, to compile source-analyte signal sequences for the annotated dataset from the signal sequences of other analyte types which agglomerate more frequently.

When compiling source-analyte signal sequences, image signals from source-analyte signal sequences of other analyte types are compiled such that they result precisely in a source-analyte signal sequence with the particular order of colored and uncolored signals according to the codebook 210 for the respective analyte types which agglomerate only rarely.

After the signal sequences for the annotated dataset have been read out from the source data, the annotated dataset is provided for training the machine learning model.

According to one configuration of the first and second embodiments, during the recording of the spot images 221, one image stack of spot images 221 offset in height can be recorded, respectively. The spot images 221 of the image stacks offset in height are preferably already registered to one another during the recording of the image stack. Accordingly, the source images 222 are also recorded for the spot images 221 such that in each case one image stack of source images 222 offset in height is recorded. The images of the image stack of source images 222 are preferably also recorded such that they are registered to one another.

If image stacks are recorded when recording the source images 222 and the spot images 221, not only must the images of an image stack be registered with one another, but also the images of image stacks of different coloring rounds.

Further possible implementations of the result class assignment model comprise a binarization model, an embedding model or a semantic segmentation model.

Embedding models are configured such that they perform mapping into an embedding space, wherein both the source signal sequences and the target bit sequences map into an embedding space. The mapping into the embedding space is executed precisely such that a distance between an embedding of the target bit sequence of the codebook 210 of an analyte type, referred to as the target embedding of the analyte type, and an embedding of the source signal sequence of the analyte type, referred to as the source analyte embedding, in the embedding space is as small as possible, and a distance between embeddings of different analyte types and a distance between embeddings of background signal sequences and analyte signal sequences is as large as possible. This is achieved in particular by using special objective functions in the training.

In the inference, a result class is then determined on the basis of a minimum distance of an embedding of a recorded signal sequence of target embeddings of the respective target bit sequences of the analyte types.

A semantic segmentation model outputs a segmentation mask of an input analyte image sequence, in which segmentation mask a result class is assigned to each image region of an input analyte image sequence. FIG. 3 shows by way of example a plurality of such segmentation masks 203, in particular a segmentation mask 203 (a) in which the cell borders are highlighted, a segmentation mask 203 (b) in which the spot image regions 223 are highlighted, and a segmentation mask 203 (c) in which the background image regions are illustrated hatched. Correspondingly, an input datum comprises an image sequence, in training correspondingly the source analyte image sequence, in the output datum a result class is assigned to each image region of the input analyte image sequence. A target output in the annotated dataset is correspondingly a segmentation mask in which a result class is assigned to each image region. In particular, it would also be sufficient during the training if a result class, that is to say correspondingly an analyte type, is assigned to all spot image regions of the source analyte image sequence, and all image regions without an assigned result class are interpreted as background class.

According to a further configuration, the result class assignment model is configured as a binarization model. The binarization model is designed such that it identifies the colored signals and the uncolored signals in the source signal sequences. The result class assignment model accordingly learns on the basis of the source signal sequences the determined ratio of colored signal to uncolored signal, of colored signal to colored signal, of uncolored signal to colored signal or of uncolored signal to uncolored signal and accordingly assigns a value of colored signal or uncolored signals to the input image signals.

The binarization model maps the image signals of an analyte signal sequence, in particular of the source-analyte signal sequences which comprise colored signals and the uncolored signals, to bit values, that is to say “true” and “false”. In a training of the binarization model, the source signal sequences are mapped onto bit sequences.

An output of the binarization model is in particular a binarized source signal sequence, also referred to as source bit sequence. On the basis of the binarized source signal sequences, an analyte type or a result class of the respective source signal sequence can be determined by means of matching with the codebook 210.

That is to say, an annotated dataset comprises as the input data of the binarization model the source signal sequences, and as target data the target bit sequences of the codebook 210 corresponding to the assigned analyte type.

Alternatively, the binarization model can also be designed such that a soft assignment is output which outputs a probability of being a colored signal for each image signal in the signal sequence.

The binarizing of the signal sequences can also be carried out using a heuristic approach. Alternatively, a generative model can also perform the mapping into the binary space.

The generative model used can be, for example, one of the following models: an active appearance model (AAMs), a generative adversarial network (GANs), a variational autoencoder (VAEs), an auto-regressive model or a diffusion model.

According to one configuration of the second embodiment, the machine learning model can additionally be a candidate extraction model or a registration model. The input and target data contained in the annotated dataset differ for such models from the annotated dataset for a result class assignment model as follows.

If the machine learning model is a candidate extraction model, the machine learning model is trained to extract or read out or filter out the signal sequences of analytes from an analyte image sequence for a further processing of the signal sequences of the analytes. As above with reference to the result class assignment model, the model can be trained as a classification model to assign one of the classes analyte signal sequence or non-analyte signal sequence, also referred to as background signal sequence, to each signal sequence or each image region of an analyte image sequence. That is to say, the result class assignment model differs from the candidate extraction model in that the input signal sequences are assigned to only two different result classes.

Correspondingly, the input data in the annotated dataset comprise source signal sequences, in particular source-analyte signal sequences and source-background signal sequences. The target data in the annotated dataset comprise information about whether the respective input data are to be assigned to the source-analyte signal sequences or to the source-background signal sequences. An exact implementation of the candidate extraction model can be in particular a classification model, a detection model, a semantic segmentation model as described above with reference to the result class assignment model.

In principle, the outputs of the respective implementations differ primarily in the form of the outputs, but the information contained therein is in each case the corresponding information whether the signal sequences (or the image regions) are background image regions or image regions of analytes, or whether the respective signal sequences are based on signals of background image regions or on signals of analytes.

If the machine learning model is a registration model, the machine learning model is trained to determine registration information for two or more images. The registration model can in turn be implemented in different ways.

In particular, the registration model can be implemented such that, for example, two or more images are input as input data into the registration model and the registration model registers the input images internally to one another and outputs them as images registered to one another. According to this implementation, the target data are the versions of the respectively input images of the input data registered to one another.

Alternatively, the machine learning model can also output only the registration information of the input images to one another as output data. For this case, the target data are precisely the registration information.

When providing the annotated data set according to this configuration of the second embodiment, the step S5 can be omitted, since it is irrelevant for registering the images whether and if so which analyte types are to be seen in an image.

In step S6, the images to be registered to one another have to be read out from the source data as input data for the annotated dataset. How exactly the output data or the target data of the annotated dataset are composed for the training of the registration model depends on the exact implementation of the registration model. The different possible implementations are described in more detail below and the output data of the respective implementation are then also described there in each case for the different implementations and accordingly also the target data for the training of the respective implementations.

According to a first implementation of the registration model, the registration model is implemented as a single-stage model, into which images not registered to one another are input as input data and outputs the images registered to one another without explicit output of registration information. A determination of the registration information is thus carried out completely within the registration model.

Alternatively, the single-stage model can also output the registration information comprising at least translation and rotation information of the input images with respect to one another. The registration information determined in particular in step S4 is then precisely used as target data.

In particular, in step S4, the registration information can be determined by means of an ICP algorithm based on the spot analyte image sequence, wherein in particular the spot image regions with the image signals above the spot threshold value are used as registration structure for registering the spot images 221 of the different coloring rounds with respect to one another.

According to a further alternative, the registration model can also be implemented as a registration structure identification model. The registration structure identification model is designed or trained to output only location information of registration structures identified in the images to be registered to one another instead of registration information or images registered to one another. After the output of the location information of the identified registration structures, a classic iterative method, for example an ICP algorithm, can be used to suitably align the location information of the found registration structures in the images to be registered to one another and to determine the registration information based thereon. Preferably, the model can also output a type of the identified registration structure.

The various registration structures to be identified include in particular a cell edge, cell organelles, cell nuclei, cytoskeleton, mitochondria, image regions with a brightness above a brightness threshold, in particular spot image regions, point objects, caused by markers or marker accumulations, structures with a minimum degree of image sharpness, for example edges, image regions which have a certain, in particular predetermined, structure or texture, holding frames, cover glasses, sample stages, sample holders, corrugated plates/corrugated dishes or other non-sample structures.

In particular, the registration model can also be implemented as a deep supervision registration model. Like the registration structure identification model described above, the deep supervision registration model outputs the location information of identified registration structures, but here as an intermediate output, that is to say as an output of one of the intermediate layers of an underlying neural network. The intermediate output then serves as deep supervision signal. For this case, the annotated dataset also has, in addition to the target data, intermediate target outputs which are still to be supervised, here the location information of the registration structures to be identified.

In addition, the registration model implemented as a deep supervision registration model also determines the registration information of the images to be registered to one another and input as input data. In particular, the registration information is output as output data or the images registered to one another.

The location information of the registration structures to be identified can in particular be determined before training. The possible registration structures comprise inter alia the cell border, cell organelles, cell nuclei, cytoskeleton, mitochondria, point objects, structures with a minimum degree of image sharpness, for example any desired edges, image regions which have a certain, in particular predetermined, structure or texture, holding frames, cover glasses, sample stages, sample holders, corrugated dishes or also other non-sample structures. In particular, the registration structures can be identified by a user in a semi-automatic method. Alternatively, a trained model can also be used for identifying the registration structures.

According to the respective implementation, the respective input data and target data and, if appropriate, the intermediate target outputs must correspondingly be stored in the annotated dataset.

In particular, the registration information used for training, be it output registration information or the output registered images, can be determined by means of an ICP algorithm in which the spot images 221 and the spot image regions 223 are used as registration structures. In particular, the source images 222 corresponding to the respective spot images 221 are registered to the spot images 221 owing to the type of recording; correspondingly, the source images 222 can be used as input data for training a registration model which can register images to one another in which no image signals above the spot threshold value appear; correspondingly, a registration model is trained which can register two images to one another without the particularly bright points.

In particular, the last recorded source images 222 described above can be used for training the registration model. If the last recorded source images 222 are used during the training of a registration model, the only structures on the basis of which the registration model could learn a registration would be the autofluorescent structures in the sample. In particular, the registration model could therefore be trained to identify the autofluorescent structures and to register the identified structures to one another. A registration model trained in this way would also be able to register microscope images, which only have autofluorescence signals, to one another. This would, in particular, make a prior marking of analytes with markers superfluous, which is why the registration could be accelerated and, in addition, the sample would be preserved.

According to a third embodiment, the present invention provides a method for training a machine learning model by means of an annotated dataset provided according to the second embodiment. The method comprises in particular the method for providing an annotated dataset, as well as a method for providing source data, in particular the methods according to the first and the second embodiments.

In addition, the method comprises a step S7 of training the machine learning model, comprising in particular inputting the input data of the annotated data set into the machine learning model, calculating the objective function on the basis of the annotated data set and the output of the machine learning model and optimizing an objective function by adapting model parameters of the machine learning model.

The objective function can capture, in particular, a difference between output data of the machine learning model and target data contained in the annotated dataset. However, if the machine learning model is, for example, an embedding model, the objective function captures a difference between the embedded input data and the embedded target data.

The optimization of the objective function can be carried out in particular by means of a stochastic gradient descent method and back propagation. However, any other known form of optimization is also possible.

According to step S7, the machine learning model is the result class assignment model which is trained for carrying out the result class assignment. According to step S7, the training is terminated as soon as the objective function reaches a predetermined threshold value.

According to the third embodiment, the result class assignment model can be a classifier which is trained as a complete convolutional network. The result class assignment model is initially trained as a classification model, which is a fully connected network with fully connected slices, with the source signal sequences of individual image regions, in particular of spot image regions 222. For this purpose, the memory module 131 inputs source signal sequences of the annotated dataset into the result class assignment model. The classification model assigns a result class to the source signal sequence, which result class indicates in particular an analyte type.

If the classification model is fully trained with the fully connected layers, then the fully connected layers are converted into fully convolutional layers. The resulting complete convolutional network can then process a complete analyte image sequence as input. As an output, the fully trained classification model or the network which is then converted into the fully convolutional network outputs a result class for each image region of the source image sequence.

According to one configuration, however, the classification model can also be trained as a fully convolutional network.

According to a fourth embodiment, the present invention provides a method for validating a processing model, in particular by means of an annotated dataset provided according to the second embodiment.

The method for validating a processing model comprises in particular the step of providing an annotated dataset according to the method according to the second embodiment.

The processing model to be validated can be one of the processing models described above with reference to the first to third embodiments, in particular one of the machine learning models. Alternatively, however, the processing model to be validated can also be any other possible processing model for processing analyte image sequences.

As a further step, the method comprises a step of inputting the input data of the annotated dataset into the processing model to be validated. The annotated dataset comprises, as described above with reference to the method for providing an annotated dataset, other input data depending on the type of the processing model, or on the type of the processing mapping executed by the processing model. The processing model can be, for example, a result class assignment model, a candidate extraction model or else a registration model.

In contrast to the training of a processing model, in the validation of a processing model, the output datum is compared with a target datum and, on the basis of the matching carried out, it is evaluated whether the processing model correctly executes the respective processing mapping.

For example, analyte image regions output by the processing model to be validated and analyte image regions to be identified according to target datum in the annotated dataset are compared.

Alternatively, the assigned result classes are compared with one another, further criteria in the validation of a processing model are, in particular, a number of the identified analyte image regions or a number of analyte image regions identified per analyte type, a number of image regions incorrectly identified as analyte image regions or a number of analyte image regions incorrectly assigned to the background.

Furthermore, the validation of a processing model can comprise determining a sensitivity of a processing model. According to some embodiments of the present invention, the sensitivity of a processing model indicates the extent to which a processing model is able to also identify analyte signal sequences in analyte image sequences whose image signal is offset only minimally from a background image signal. That is to say, the sensitivity of a processing model is the greater, the smaller the distance between an image signal of a colored signal in an analyte signal sequence and a background image signal.

In order to determine a sensitivity of a processing model, the validation is carried out using an annotated dataset having a plurality of source analyte image sequences. Each of the plurality of source analyte image sequences corresponds to a specific bleaching round and successive bleaching rounds have decreasing image signals in the spot image regions.

The inventors have recognized that the finding of analyte signal sequences in an analyte image sequence becomes the more difficult, the smaller the difference between colored signals and uncolored signals. If a bleaching sequence of source images 222 is stored for each coloring round for each spot image 221, a respective image sequence can be created from the source images 222 of the bleaching sequence of the different coloring rounds, wherein the colored signals of the spot image regions 223 of successive source images in the image sequences respectively having image signals falling, which is why the different successive source analyte image sequences of successive bleaching rounds also have declining image signals.

That is to say, the better a processing model to be validated can still identify analyte signal sequences or assign analyte types even in the case of lower image signals of colored signal in the spot image regions, the better the sensitivity of the validated processing model.

According to a fifth embodiment, the present invention provides a method for unsupervised learning.

The method for unsupervised learning comprises, in particular, a cluster analysis by means of Gaussian mixed model clustering. The cluster analysis is applied in particular to the source data, in particular to the source-analyte signal sequences. In this case, a cluster center should in each case be able to be determined for all source-analyte signal sequences of a particular analyte type of the codebook 210. The specific cluster centers form target cluster centers.

If new data are now recorded or signal sequences of non-agglomerating analytes are input into a processing model executing the cluster analysis, then the analyte signal sequences of non-agglomerating analytes should be mapped into the vicinity of the target cluster centers, such that an analyte type of an analyte signal sequence can be determined on the basis of a smallest distance from a target cluster center.

In addition to the source-analyte signal sequences used when carrying out the cluster analysis, source-background signal sequences can also be used, which should, as far as possible, not coincide with the target cluster centers, such that the background signal sequences can be separated well from the analyte signal sequences.

As an alternative to Gaussian mixed model clustering, the clustering method can also be an abovementioned cluster clustering, a kernel-based principal component analysis clustering, an E.M. clustering, a Leiden clustering, a Lowe clustering or a device of a list clustering.

According to a sixth embodiment, the present invention provides a source data acquisition system 1, wherein the source data acquisition system 1 comprises the microscope 100 and the evaluation device 130.

According to a seventh embodiment, the present invention provides an evaluation device 130, wherein the machine learning model of the evaluation device 130 has been trained according to the method for training a machine learning model for processing analyte image sequences as described above.

Examples of the devices, systems, and/or methods of various embodiments are provided below. An embodiment of the devices, systems, and/or methods can include any one or more, and any combination of, the examples described below.

Example 1 is a method for providing source data for training or validating a processing model for processing analyte image sequences, wherein an analyte image sequence is generated by labeling analytes with markers in a plurality of coloring rounds and detecting the markers with a camera, in particular the markers are selected based on a codebook such that signal sequences of analytes in an image region over the analyte image sequence comprise colored signals and uncolored signals, the camera captures an image of the analyte image sequence in each coloring round, comprising:
capturing a spot analyte image sequence of a sample,
providing an evaluation of image regions based on image signals of the image regions,
identifying one or more spot image regions in the spot images from the evaluation of the image regions, wherein the evaluation of spot image regions of spot images for colored signals has a spot evaluation, characterized in that the method further comprises:
capturing one or more source analyte image sequences comprising source images registered to the spot images, comprising reducing a scene contrast in order to record the source images registered to the spot images with a scene contrast reduced compared to the spot images, such that the evaluation of the spot image regions based on the image signals of the spot image regions for the source images of at least one of the source analyte image sequences for colored signals has a non-spot evaluation, and
providing the at least one of the source analyte image sequences as source data.

Example 2 includes the subject-matter of any one or more of the preceding Examples, wherein the recording of analyte image sequences of the sample comprises exciting a fluorescence of the markers by means of a light source and comprises detecting a fluorescence signal emitted by the excited markers.

Example 3 includes the subject-matter of any one or more of the preceding Examples, wherein the reducing of a scene contrast comprises bleaching one or more spot subarea of the sample corresponding to the spot image regions.

Example 4 includes the subject-matter of Example 3, wherein the bleaching comprises illuminating at least the spot subareas of the sample multiple times, in particular recording a plurality of the source analyte image sequences and/or applying a chemical fluorescence suppressor.

Example 5 includes the subject-matter of Example 4, wherein a number of illuminations in the illuminating multiple times is a predefined number of illuminations, the number of recorded source analyte image sequences is a predefined number of recorded source analyte image sequences or the number of illuminations and the number of recorded source analyte image sequences is determined from the image signals of the spot image regions and is determined in particular based on the image signals of the spot image regions, the evaluation and/or an average background image signal.

Example 6 includes the subject-matter of any one or more of the preceding Examples 3 to 5, wherein the bleaching comprises matching image signals of an autofluorescence of background image regions surrounding the spot image regions and the image signals of the spot image regions, such that the image signals of the background image regions are reduced less strongly than the image signals of the spot image regions.

Example 7 includes the subject-matter of the preceding Example 6, wherein the matching in particular comprises one of:
selecting a temporal distance between two successive illuminations such that the image signals of the autofluorescence regenerate between two successive illuminations,
illuminating the sample during the bleaching such that only the spot subareas of the sample are illuminated, wherein in particular the light source is a laser and an imaging device is a laser scanning microscope, LSM,
processing the image signals of the background image regions such that the image signals of the background image regions remain approximately constant over all illuminations,
selecting a concentration of the fluorescence suppressor such that the image signals of colored signals of spot image regions approach the image signals of the background image regions.

Example 8 includes the subject-matter of any one or more of the preceding Examples 3 to 7, wherein the capturing of the source analyte image sequence comprises using a source configuration of the imaging device, wherein the source configuration in particular comprises one or more of the parameters of a number of illuminations, an illumination intensity, the illuminated image regions and a regeneration time between two illuminations, wherein the parameters are selected such that a scene contrast of the source analyte image sequence is reduced compared to the spot analyte image sequence.

Example 9 includes the subject-matter of any one or more of the preceding Examples 1 or 2, wherein the reducing of a scene contrast comprises changing the spectral properties of an imaging device, in particular the recording of the source analyte image sequence comprises using a source configuration of the imaging device, wherein the source configuration has changed spectral properties compared to a spot configuration of the imaging device used during the recording of the spot analyte image sequence, such that a scene contrast of the source analyte image sequence is reduced compared to the spot analyte image sequence.

Example 10 includes the subject-matter of Example 9, wherein the source configuration and the spot configuration are different for different ones of the coloring rounds, in particular the spot configuration and the source configuration are dependent on a marker to be detected in the respective coloring round.

Example 11 includes the subject-matter of any one or more of the preceding Examples 9 or 10, wherein the source configuration and the spot configuration differ in one or more of the following spectral properties:
an illumination spectrum of the light source,
a spectral transmittance of a light source filter for filtering the illumination spectrum emitted by the light source,
a spectral transmittance of a fluorescence filter for filtering the fluorescence signal of the markers, and
a spectral transmittance and/or a spectral reflectivity of a dichroic mirror, wherein the spectral transmittance indicates a spectral component which is reflected and/or transmitted by the dichroic mirror.

Example 12 includes the subject-matter of any one or more of the preceding Examples 9 to 11, wherein a source illumination spectrum used according to the source configuration has a lower overlap with the excitation spectrum of the marker to be excited than a spot illumination spectrum of the light source used according to the spot configuration, such that a scene contrast of the source analyte image sequence is reduced compared to the spot analyte image sequence, wherein in particular the image signals of the spot image regions of the source images are reduced compared to the image signals of the spot image regions of the spot images.

Example 13 includes the subject-matter of any one or more of the preceding Examples 9 to 12, wherein a source light source filter for filtering the illumination spectrum of the light source used according to the source configuration filters the illumination spectrum of the light source such that the resulting filtered illumination spectrum has a lower overlap with the excitation spectrum of the marker to be excited than an illumination spectrum filtered with a spot light source filter used according to the spot configuration, such that a scene contrast of the source analyte image sequence is reduced compared to the spot analyte image sequence, wherein in particular the image signals of the spot image regions of the source images are reduced compared to the image signals of the spot image regions of the spot images.

Example 14 includes the subject-matter of any one of the preceding Examples 9 to 13, wherein a source fluorescence filter used according to the source configuration has a worse overlap with the fluorescence spectrum of the excited marker than a spot fluorescence filter used according to the spot configuration, such that a scene contrast of the source analyte image sequence is reduced compared to the spot analyte image sequence, wherein in particular the image signals of the spot image regions of the source images are reduced compared to the image signals of the spot image regions of the spot images.

Example 15 includes the subject-matter of any one or more of the preceding Examples 9 to 14, wherein the spectral properties of a dichroic source mirror used according to the source configuration and dichroic spot mirror used according to the spot configuration are selected such that the source mirror has a worse match with the illumination spectrum, the excitation spectrum of the marker to be excited and/or the fluorescence spectrum of the excited marker than the spot mirror, such that a scene contrast of the source analyte image sequence is reduced compared to the spot analyte image sequence, wherein in particular the image signals of the spot image regions in the source analyte image sequence are reduced compared to the image signals of the spot image regions of the spot analyte image sequence.

Example 16 includes the subject-matter of any one or more of the preceding Examples, wherein the reducing of the scene contrast comprises unequally illuminating the sample for capturing the source images, wherein spot subareas of the sample mapping to the spot image regions and image regions surrounding the spot subareas are just illuminated unequally such that the scene contrast is reduced.

Example 17 includes the subject-matter of Example 16, wherein the unequal illuminating comprises a stronger illumination of the image regions surrounding the spot subareas, a weaker illumination of the spot subareas of the sample or both.

Example 18 includes the subject-matter of the preceding Examples 17, wherein the light source is in particular a laser during the unequal illumination and the unequal illumination comprises controlling the laser such that the image regions of the sample are illuminated evenly during the recording of the spot images and the unequal illumination is carried out during the recording of the source images.

Example 19 includes the subject-matter of any one or more of the preceding Examples 3 to 18, wherein the recording of the source images comprises one or more of the bleaching according to claim 3, the changing of the spectral properties according to claim 9, and the unequal illumination according to claim 16.

Example 20 includes the subject-matter of any one or more of the preceding Examples, wherein the providing of an evaluation of the spot image regions comprises one or more of:
matching the image signals of image regions of a spot image with a spot threshold value and evaluating image regions with the spot evaluation if image regions have image signals above the spot threshold value,
Using classic image processing methods for feature acquisition in images, in particular for detecting blobs, for example with a blob detector, a Viola Jones detector or a histogram of oriented gradient detector, HoG detector,
inputting the image signals of image regions of at least one spot image into a candidate identification model, wherein the candidate identification model outputs the evaluation for each image region.

Example 21 includes the subject-matter of the preceding Example 20, further comprising determining the spot threshold value, in particular based on an average image signal in the spot image, based on the image region to be evaluated and an average image signal in a surrounding of the image region, based on context information or based on a coloring round.

Example 22 includes the subject-matter of any one or more of the preceding Examples 20 or 21, wherein the candidate identification model is designed as an evaluation model which has been trained to evaluate the image signals, wherein image signals of image regions of a spot image with an evaluation above the spot threshold value are identified as the spot image regions, wherein the candidate identification model takes into account in particular the image signals of the image region to be evaluated and the image signals of image regions surrounding the image region to be evaluated in the evaluation.

Example 23 includes the subject-matter of any one or more of the preceding Examples 20 to 22, wherein the candidate identification model has also been trained to determine the spot threshold value from the image signals of the spot image.

Example 24 includes the subject-matter of any one or more of the preceding Examples 22 or 23, wherein a number of illuminations in the bleaching for reducing the scene contrast can be determined from an evaluation output by the candidate identification model.

Example 25 includes the subject-matter of any one or more of the preceding Examples 20 to 24, wherein the candidate identification model is designed as a detection model, wherein the detection model has been trained to output a list of the spot image regions.

Example 26 includes the subject-matter of any one or more of the preceding Examples 20 to 24, wherein the candidate identification model is designed as a classification model, wherein the classification model has been trained to assign a class to input image signals of image regions, wherein assigned classes comprise at least one class of spot image region and non-spot image region.

Example 27 includes the subject-matter of any one or more of the preceding Examples 20 to 24, wherein the candidate identification model is designed as an image-to-image model which has been trained to assign a probability of being a spot image region to each image region.

Example 28 includes the subject-matter of any one or more of the preceding Examples, further comprising determining the spot configuration and the source configuration for recording the spot images and the source images by means of a configuration determination model.

Example 29 includes the subject-matter of the preceding Example 28, wherein the input data of the configuration determination model comprise context information of the spot analyte image sequence to be recorded, in particular information about an excitation spectrum of markers used, an emission spectrum of the markers used, as well as available optical components, in particular light sources, light source filters, fluorescence filters and/or dichroic mirrors.

Example 30 includes the subject-matter of the preceding Examples 29, wherein the determining of the source configuration comprises inputting the spot images into the configuration determination model, wherein the configuration determination model has been trained to determine the source configuration for recording the source image based on an input spot image, wherein the source configuration comprises one or more of the following information:
a light source used,
a light source filter used,
a fluorescence filter used,
a dichroic mirror used,
an illuminance of the light source,
an illumination time of the light source,
a number of illuminations carried out,
a number of recorded source images,
a temporal distance between successive illuminations,
a size and/or position of an illuminated subarea of the sample, and
a concentration of a chemical fluorescence suppressor.

Example 31 includes the subject-matter of the preceding Example 30, wherein the configuration determination model further takes into account context information in the determining of the source configuration.

Example 32 includes the subject-matter of any one or more of the preceding Examples 30 or 31, wherein the determining of a source configuration further comprises:
inputting a source image into the configuration determination model and determining, by the configuration determination model, the source configuration for further source images to be recorded based on the input source image or based on the input source image and the spot image.

Example 33 includes the subject-matter of any one or more of the preceding Examples, further comprising:
reading out candidate analyte signal sequences from spot image regions of the spot analyte image sequence, wherein signal sequences of image regions for each coloring round comprise image signals of the image regions of the image of the respective coloring round,
determining a result class of the candidate analyte signal sequences, wherein the result classes comprise at least one class for each analyte type to be identified, and the determination of the result class is carried out on the basis of a codebook.

Example 34 includes the subject-matter of the preceding Example 33, wherein the result classes further comprise a background class for candidate analyte signal sequences, which cannot be assigned to any of the analyte types to be identified, and in particular candidate analyte signal sequences assigned to the background class are identified as non-analyte signal sequences, and the candidate analyte signal sequences assigned to one of the analyte types are identified as analyte signal sequences.

Example 35 includes the subject-matter of any one or more of the preceding Examples, wherein the method further comprises determining registration information of images of different coloring rounds with respect to one another, wherein the registration information comprises at least one of translation information and rotation information of the images of the different coloring rounds with respect to one another.

Example 36 includes the subject-matter of the preceding Example 35, wherein the registration information is determined based on the spot images of the different coloring rounds or the registration information is determined based on the spot images and a part of the source images of the different coloring rounds.

Example 37 includes the subject-matter of the preceding Example 36, wherein the registration information is the same for the spot images and the source images.

Example 38 includes the subject-matter of any one or more of the preceding Examples 35 to 37, wherein the determining of the registration information further comprises:
inputting the images of the different coloring rounds into a registration model, wherein the registration model has been trained to identify registration structures in the images, to determine the registration information or to identify the registration structures and to determine the registration information based on the registration structures.

Example 39 is a method of providing an annotated dataset for training a processing model for processing analyte image sequences comprising:
selecting input data for inputting into a processing model from source data, wherein the source data has been recorded according to the method according to any one of the preceding examples,
selecting target data corresponding to the input data, wherein the target data is determined depending on the processing model to be trained, and
providing the input data and the selected target data as annotated data set.

Example 40 includes the subject-matter of the preceding Example 39, wherein the processing comprises determining registration information of at least two images of different coloring rounds of an analyte image sequence at least partially capturing the same parts of the sample and the processing model is in particular a registration model, the selecting of input data comprises selecting at least two source images of a source analyte image sequence of the source data and the selecting of target data in particular comprises reading out registration information from the source data or the selecting of target data comprises determining registration information for spot images corresponding to the at least two source images of a spot analyte image sequence corresponding to the source analyte image sequence and selecting the registration information as target data.

Example 41 includes the subject-matter of Example 40, wherein image signals of image regions of selected source images rest above a background threshold value.

Example 42 includes the subject-matter of any one or more of the preceding Examples 39 to 41, wherein the target data further comprises location information of registration structures to be identified.

Example 43 includes the subject-matter of any one or more of the preceding Examples 39 to 42, wherein the processing comprises extracting candidate signal sequences from an analyte image sequence and the processing model is in particular a candidate extraction model, the input data comprises signal sequences of spot image regions over at least one of the source analyte image sequences, the candidate extraction model is implemented in particular as a classification model, semantic segmentation model, detection model or as an image-to-image model, in particular a semantic segmentation model, so that candidate signal sequences can be read out from an analyte image sequence on the basis of an output of the candidate extraction model, and the target data is determined according to the implemented candidate extraction model.

Example 43 includes the subject-matter of any one or more of the preceding Examples 39 to 42, wherein the processing comprises assigning signal sequences to a result class and the processing model is in particular a result class assignment model, the input data comprises signal sequences of image regions over one of the source analyte image sequences, the result classes comprise at least one class for each analyte type to be identified and a background class, the result class assignment model can be implemented in different implementations, in particular as a classification model, semantic segmentation model, detection model, binarization model or as an embedding model, wherein the classification model is trained to output a result class or a probability distribution over the possible result classes, the semantic segmentation model is trained to output a result class for each image region, the detection model is trained to acquire a list of image regions of the analytes and to output the respectively determined analyte type for the image regions, the binarization model is trained to output a binarized signal sequence, wherein the binarization model assigns a binary value to the image signals of a signal sequence, which binary value corresponds to either a colored or an uncolored signal, so that the analyte type and thus the result class can be determined by a matching of the binarized signal sequence with target signal sequences of a codebook, the embedding model embeds input signal sequences into an embedding space such that the analyte type and thus the result class can be determined on the basis of a distance to a nearest target embedding of target signal sequences of the analyte types to be identified, and the target output is selected according to the implementation of the result class assignment model.

Example 45 includes the subject-matter of the preceding Example 44, wherein the selection of the input data is carried out on the basis of the determined target data such that a sufficient number of input data is present in the annotated data set for all result classes to be identified.

Example 46 includes the subject-matter of any one or more of the preceding Examples 45, wherein the selecting of input data for the annotated data set is carried out on the basis of the determined result classes of the signal sequences of the spot analyte image sequence and the input data is selected from the source analyte image sequence.

Example 47 includes the subject-matter of any one or more of the preceding Examples 39 to 46, wherein the input data comprises, depending on the processing to be trained, one or more of:
the source analyte image sequence,
the spot analyte image sequence,
images or fields of view of the source analyte image sequence,
one or more source signal sequences from the source analyte image sequence, wherein the source signal sequences are signal sequences of spot image regions of the source images over the coloring rounds,
one or more background signal sequences, wherein the background signal sequences comprise signal sequences of background image regions, signal sequences of image signals randomly selected from the source analyte image sequence and signal sequences of image signals randomly selected from an unlabeled analyte image, the background image regions are image regions that capture no image signals of analytes, and the unlabeled analyte images are images in which analytes are not labeled with markers and image regions of the unlabeled analyte images capture autofluorescence signals of the sample.

Example 48 includes the subject-matter of any one or more of the preceding Examples 39 to 47, wherein context information is included in the determining of the target data.

Example 49 includes the subject-matter of any one or more of the preceding Examples, wherein context information is included in the recording of the spot analyte image sequence.

Example 50 includes the subject-matter of any one or more of the preceding Examples, wherein context information is included in the identifying of spot image regions.

Example 51 includes the subject-matter of any one or more of the preceding Examples, wherein context information is included in the reducing of a scene contrast.

Example 52 is a method for training a machine learning system having a processing model for processing analyte image sequences, wherein an analyte image sequence is generated by labeling analytes with markers in a plurality of coloring rounds and detecting the markers with a camera, the camera captures an image of the analyte image sequence in each coloring round, the markers are selected according to a codebook such that image signals of an analyte in an image region over the analyte image sequence comprise colored signals and uncolored signals in an order predefined according to the codebook, comprising:
providing an annotated data set,
optimizing an objective function by adapting the model parameters of the processing model, wherein the objective function captures a difference between an output datum output by the processing model on the basis of an input datum of the annotated data set and a target datum of the annotated data set corresponding to the input datum, characterized in that the annotated data set was generated by means of the method according to the Examples 39 to 51.

Example 53 is a method for training a machine learning system having a processing model for processing analyte image sequences, wherein an analyte image sequence is generated by labeling analytes with markers in a plurality of coloring rounds and detecting the markers with a camera, the camera captures an image of the analyte image sequence in each coloring round, the markers are selected according to a codebook such that image signals of an analyte in an image region over the analyte image sequence comprise colored signals and uncolored signals in an order predefined according to the codebook, comprising:
providing a training data set,
carrying out a cluster analysis algorithm with the processing model on the basis of the training data set, characterized in that the training data set is based on the source data generated by means of the method according to Examples 1 to 38, in particular on the signal sequences of spot image regions of the source analyte image sequence.

Example 54 is an annotated dataset for use in a method for training a machine learning system comprising a processing model, wherein the annotated dataset is used in a method according to Examples 52 or 53.

Example 55 is a method for validating a processing model for processing an analyte image sequence, wherein the image sequence is generated by labeling analytes with markers in a plurality of coloring rounds and detecting the markers with a camera, the camera captures an image of the image sequence in each coloring round, the markers are selected such that signal sequences of image regions that capture image signals of an analyte comprise colored signals and uncolored signals over the image sequence, comprising:
providing an annotated data set for validating the processing model,
inputting the annotated data set into the processing model,
matching an output datum output by the processing model on the basis of an input datum of the annotated data set with the target datum corresponding to the input datum, characterized in that the annotated data set was generated by means of the method according to Examples 39 to 51.

Example 56 includes the subject-matter of Example 55, wherein the matching of the output datum with the target datum comprises one or more of:
matching of correctly identified analyte image regions,
matching of locations assigned to analyte image regions,
number of identified analyte image regions or analyte image regions identified per analyte type,
number of image regions incorrectly identified as analyte image region,
number of analyte image regions incorrectly assigned to the background.

Example 57 includes the subject-matter of any one or more of the preceding Examples 55 or 56, wherein the validating comprises comparing a plurality of different processing models or processing models trained with different annotated data sets with one another.

Example 58 includes the subject-matter of any one or more of the preceding Examples 55 to 57, wherein the annotated dataset comprises a plurality of source analyte image sequences, wherein each of the plurality of source analyte image sequences corresponds to a bleaching round and the colored signals of spot image regions have image signals decreasing in successive bleaching rounds, and a sensitivity of the processing model to be validated is determined on the basis of the plurality of source analyte image sequences depending on the image signals in the spot image regions.

Example 59 is a computer program product comprising instructions which, when the program is executed by a computer, cause the latter to carry out the method according to any one of the preceding Examples 1 to 58, the computer program product being in particular a computer-readable storage medium.

Example 60 is an evaluation device for evaluating images of an analyte image sequence, comprising means for carrying out the method according to any of the preceding Examples 1 to 59.

Example 61 is an image generation device for capturing microscope images, comprising an evaluation device for evaluating images of an analyte image sequence comprising means for carrying out the method according to any one of the preceding Examples 1 to 58.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for providing source data for training or validating a processing model for processing analyte image sequences, wherein an analyte image sequence is generated by labeling analytes with markers in a plurality of coloring rounds and detecting the markers with a camera, the markers are selected based on a codebook such that signal sequences of analytes in an image region over the analyte image sequence comprise colored signals and uncolored signals, the camera captures an image of the analyte image sequence in each coloring round, the method comprising:
capturing a spot analyte image sequence of a sample,
providing an evaluation of image regions based on image signals of the image regions,
identifying one or more spot image regions in the spot images from the evaluation of the image regions, wherein the evaluation of spot image regions of spot images for colored signals has a spot evaluation;
capturing one or more source analyte image sequences comprising source images registered to the spot images, comprising reducing a scene contrast in order to record the source images registered to the spot images with a scene contrast reduced compared to the spot images, such that the evaluation of the spot image regions based on the image signals of the spot image regions for the source images of at least one of the source analyte image sequences for colored signals has a non-spot evaluation, and
providing the at least one of the source analyte image sequences as source data.

2. The method according to claim 1, wherein the reducing of a scene contrast comprises bleaching one or more spot subarea of the sample corresponding to the spot image regions, wherein the bleaching comprises one or more of illuminating at least the spot subareas of the sample multiple times, recording a plurality of the source analyte image sequences and applying a chemical fluorescence suppressor.

3. The method according to claim 2, wherein a number of illuminations in the illuminating multiple times is a predefined number of illuminations, the number of recorded source analyte image sequences is a predefined number of recorded source analyte image sequences or the number of illuminations and the number of recorded source analyte image sequences is determined from the image signals of the spot image regions and is determined based on the image signals of the spot image regions, the evaluation and/or an average background image signal.

4. The method according to claim 1, wherein the reducing of a scene contrast comprises changing the spectral properties of an imaging device, the recording of the source analyte image sequence comprises using a source configuration of the imaging device, wherein the source configuration has changed spectral properties compared to a spot configuration of the imaging device used during the recording of the spot analyte image sequence, such that a scene contrast of the source analyte image sequence is reduced compared to the spot analyte image sequence, wherein the source configuration and the spot configuration differ in one or more of the following spectral properties:
an illumination spectrum of the light source,
a spectral transmittance of a light source filter for filtering the illumination spectrum emitted by the light source,
a spectral transmittance of a fluorescence filter for filtering the fluorescence signal of the markers, and
a spectral transmittance and/or a spectral reflectivity of a dichroic mirror, wherein the spectral transmittance indicates a spectral component which is reflected and/or transmitted by the dichroic mirror.

5. The method according to the preceding claim 4, wherein the source configuration and the spot configuration are different for different ones of the coloring rounds, the spot configuration and the source configuration are dependent on a marker to be detected in the respective coloring round.

6. The method according to claim 1, wherein the reducing of the scene contrast comprises unequally illuminating the sample for capturing the source images, wherein spot subareas of the sample mapping to the spot image regions and image regions surrounding the spot subareas are just illuminated unequally such that the scene contrast is reduced, wherein the unequal illuminating comprises a stronger illumination of the image regions surrounding the spot subareas, a weaker illumination of the spot subareas of the sample or both, and the light source is a laser during the unequal illumination and the unequal illumination comprises controlling the laser such that the image regions of the sample are illuminated evenly during the recording of the spot images and the unequal illumination is carried out during the recording of the source images.

7. The method according to claim 1, wherein the providing of an evaluation of the spot image regions comprises one or more of:
matching the image signals of image regions of a spot image with a spot threshold value and evaluating image regions with the spot evaluation if image regions have image signals above the spot threshold value,
using classic image processing methods for feature acquisition in images, in particular for detecting blobs, for example with a blob detector, a Viola Jones detector or a histogram of oriented gradient detector, HoG detector,
inputting the image signals of image regions of at least one spot image into a candidate identification model, wherein the candidate identification model outputs the evaluation for each image region.

8. The method according to claim 1, further comprising determining the spot configuration and the source configuration for recording the spot images and the source images by means of a configuration determination model.

9. The method according to claim 8, wherein the determining of the source configuration comprises inputting the spot images into the configuration determination model, wherein the configuration determination model has been trained to determine the source configuration for recording the source image based on an input spot image, wherein the source configuration comprises one or more of the following information:
a light source used,
a light source filter used,
a fluorescence filter used,
a dichroic mirror used,
an illuminance of the light source,
an illumination time of the light source,
a number of illuminations carried out,
a number of recorded source images,
a temporal distance between successive illuminations,
a size and/or position of an illuminated subarea of the sample, and
a concentration of a chemical fluorescence suppressor.

10. The method according to claim 1, further comprising:
reading out candidate analyte signal sequences from spot image regions of the spot analyte image sequence, wherein signal sequences of image regions for each coloring round comprise image signals of the image regions of the image of the respective coloring round,
determining a result class of the candidate analyte signal sequences, wherein the result classes comprise at least one class for each analyte type to be identified, and the determination of the result class is carried out on the basis of a codebook.

11. The method according to claim 1, wherein the method further comprises determining registration information of images of different coloring rounds with respect to one another, wherein the registration information comprises at least one of translation information and rotation information of the images of the different coloring rounds with respect to one another, and the registration information is determined based on the spot images of the different coloring rounds or the registration information is determined based on the spot images and a part of the source images of the different coloring rounds.

12. A computer program product comprising instructions which, when the program is executed by a computer, cause the latter to carry out the method according to claim 1.

13. An evaluation device for evaluating images of an analyte image sequence, comprising means for carrying out the method according to claim 1.

14. An image generation device for capturing microscope images, comprising an evaluation device for evaluating images of an analyte image sequence comprising means for carrying out the method according to claim 1.

15. A method for training a machine learning system having a processing model for processing analyte image sequences, wherein an analyte image sequence is generated by labeling analytes with markers in a plurality of coloring rounds and detecting the markers with a camera, the camera captures an image of the analyte image sequence in each coloring round, the markers are selected according to a codebook such that image signals of an analyte in an image region over the analyte image sequence comprise colored signals and uncolored signals in an order predefined according to the codebook, comprising:
providing a training data set,
carrying out a cluster analysis algorithm with the processing model on the basis of the training data set, wherein the training data set is based on the source data generated by means of the method according to claim 1.

16. A method of providing an annotated dataset for training a processing model for processing analyte image sequences comprising:
selecting input data for inputting into a processing model from source data, wherein the source data has been recorded according to the method according to claim 1,
selecting target data corresponding to the input data, wherein the target data is determined depending on the processing model to be trained, and
providing the input data and the selected target data as annotated data set.

17. The method according to claim 16, wherein the input data comprises, depending on the processing to be trained, one or more of:
the source analyte image sequence,
the spot analyte image sequence,
images or fields of view of the source analyte image sequence,
one or more source signal sequences from the source analyte image sequence, wherein the source signal sequences are signal sequences of spot image regions of the source images over the coloring rounds,
one or more background signal sequences, wherein the background signal sequences comprise signal sequences of background image regions, signal sequences of image signals randomly selected from the source analyte image sequence and signal sequences of image signals randomly selected from an unlabeled analyte image, the background image regions are image regions that capture no image signals of analytes, and the unlabeled analyte images are images in which analytes are not labeled with markers and image regions of the unlabeled analyte images capture autofluorescence signals of the sample.

18. A method for training a machine learning system having a processing model for processing analyte image sequences, wherein an analyte image sequence is generated by labeling analytes with markers in a plurality of coloring rounds and detecting the markers with a camera, the camera captures an image of the analyte image sequence in each coloring round, the markers are selected according to a codebook such that image signals of an analyte in an image region over the analyte image sequence comprise colored signals and uncolored signals in an order predefined according to the codebook, the method comprising:
providing an annotated data set,
optimizing an objective function by adapting the model parameters of the processing model, wherein the objective function captures a difference between an output datum output by the processing model on the basis of an input datum of the annotated data set and a target datum of the annotated data set corresponding to the input datum, wherein the annotated data set was generated by means of the method according to claim 16.

19. A method for validating a processing model for processing an analyte image sequence, wherein the image sequence is generated by labeling analytes with markers in a plurality of coloring rounds and detecting the markers with a camera, the camera captures an image of the image sequence in each coloring round, the markers are selected such that signal sequences of image regions that capture image signals of an analyte comprise colored signals and uncolored signals over the image sequence, comprising:
providing an annotated data set for validating the processing model,
inputting the annotated data set into the processing model,
matching an output datum output by the processing model on the basis of an input datum of the annotated data set with the target datum corresponding to the input datum, wherein the annotated data set was generated by means of the method according to claim 16.

20. The method according to claim 19, wherein the matching of the output datum with the target datum comprises one or more of:
matching of correctly identified analyte image regions,
matching of locations assigned to analyte image regions,
number of identified analyte image regions or analyte image regions identified per analyte type,
number of image regions incorrectly identified as analyte image region,
number of analyte image regions incorrectly assigned to the background.
